(19) United States
(12) Patent Application Publication
(10) Pub. No.: US 2014/0273147 A1
(43) Pub. Date: Sep. 18, 2014

(54) METHOD FOR SYNTHESIZING METAL OXIDE PARTICLES

(71) Applicant: UT-Battelle, LLC, Oak Ridge, TN (US)

(72) Inventors: Pooran Chandra Joshi, Knoxville, TN (US); Chad E. Duty, Knoxville, TN (US); Gerald Earle Jellison, Jr., Oak Ridge, TN (US); Ilia N. Ivanov, Knoxville, TN (US); Beth Louise Armstrong, Clinton, TN (US); Ji-Won Moon, Oak Ridge, TN (US); Hyunsung Jung, Nowon-gu (KR); Adam Justin Rondinone, Knoxville, TN (US); Tommy Joe Phelps, Knoxville, TN (US)

(21) Appl. No.: 14/205,699

(22) Filed: Mar. 12, 2014

Related U.S. Application Data
(60) Provisional application No. 61/777,012, filed on Mar. 12, 2013.

(51) Int. Cl. C12P 3/00

(52) U.S. Cl. CPC C12P 3/00
USPC 435/168

(57) ABSTRACT

The invention is directed to a method for producing metal oxide particles, the method comprising subjecting non-oxide metal-containing particles to an oxidation step that converts the non-oxide metal-containing particles to said metal oxide particles. The invention is also directed to the resulting metal oxide compositions. In particular embodiments, non-oxide precursor particles are produced by microbial means, and the produced non-oxide precursor particles subjected to oxidation conditions under elevated temperature conditions (e.g., by a thermal pulse) to produce metal oxide particles or a metal oxide film.

The present application claims benefit of U.S. Provisional Application No. 61/777,012, filed on Mar. 12, 2013, all of the contents of which are incorporated herein by reference.

This invention was made with government support under Prime Contract No. DE-AC05-00OR22725 awarded by the U.S. Department of Energy. The government has certain rights in the invention.

### FIELD OF THE INVENTION

The present invention relates to the field of inorganic particles, and more particularly, to such particles having a metal oxide or mixed-metal oxide composition.

### BACKGROUND OF THE INVENTION

Particles, and particularly nanoparticles, having metal oxide compositions are increasingly being used in numerous emerging applications. Some of these include the use of magnetic nanoparticles (e.g., magnetite) in magnetic refrigeration or magnetic cooling circuits. Ferrite-type nanoparticles, in particular, are being intensely studied for their use in the fields of biomedicine, optics, and electronics. Other applications include photovoltaic materials, as used, for example, in solar cell devices.

Current methods for the production of nanoscale ferrites and other oxide ceramics generally entail calcining a precursor (e.g., a carbonate) at a high temperature, and then mechanical milling the calcined product to reduce the particle size. The process is energy and time intensive, generally difficult to control, and often requires several repetitions of the process before a final product is obtained.

Chemical processes, such as precipitation and sol-gel techniques, are also known for the production of metal oxide particles. However, these processes are typically more expensive than mechanical milling, and also generally highly limited with respect to size or shape control of the resulting particles. Often, a chemical or physical reduction step is needed to convert a metal oxide precursor to a metal oxide product. In addition, these processes often require a mechanical milling step to break up agglomerates formed during the reduction process.

The microbial synthesis of metal oxide nanoparticles is known. See, for example, U.S. Pat. Nos. 6,444,453 and 7,060,473. However, there are significant problems in the microbial process as currently practiced. For example, there is the difficulty of obtaining pure nanoparticle product bereft of microbial matter. Therefore, numerous lysing or washing steps are often required. There is also the difficulty in controlling the particle size or the morphology of the nanoparticles.

### SUMMARY OF THE INVENTION

The invention is foremost directed to a convenient method for the production of metal oxide particles having any of a variety of oxide and mixed-metal oxide compositions. The method described herein can advantageously produce a wide range of metal oxide compositions at lower cost and without the burdensome complexities of existing processes. The invention accomplishes this by employing a process in which non-oxide metal-containing particles (e.g., of a metal chalcogenide or metal pnictide composition) function as oxidizable precursors in an oxidation process. In the oxidation process, the non-oxide precursors become converted to particles having a metal oxide or mixed-metal oxide composition. As the non-oxide precursors can be produced by relatively cost efficient and simple means (e.g., by a bacterial or abiotic process), and the oxidation process can also be practiced by simple means, the overall process described herein can achieve significant reductions in cost and labor for producing a variety of metal oxide compositions, particularly the possibility of low-cost bulk production of metal oxides that have traditionally been made in limited quantities by complex methods. Moreover, the process described herein has the capability of producing metal oxide compositions heretofore unknown or unavailable and that have rare properties and specialized utilities.

The invention is also directed to the metal oxide compositions produced by the above-described method. The metal oxide particles produced herein possess any one or more of a diverse set of properties that make them useful. Some of the properties particularly considered herein include photovoltaic, photoluminescent, light-emitting, and thermoelectric properties. Such properties make these metal oxide particles useful in one or more end applications, e.g., in photovoltaic, light-emitting, and thermoelectric devices. Other applications include oxide electrode materials, such as found in lithium ion batteries and fuel cells, as well as catalytic materials, as used in the treatment of diesel engine emissions.

In particular aspects, the metal oxide particles are useful as photoluminescent-tunable materials, which find particular use in photovoltaic devices. Other types of devices that can benefit from such tunable materials include light-emitting and laser diodes. Accordingly, the method and compositions of the invention can greatly advance several types of devices, including photovoltaic devices.

### BRIEF DESCRIPTION OF THE DRAWINGS

FIG. 1. XRD patterns of microbially-produced ZnS nanocrystals (as-synthesized), and the same nanocrystals after being annealed under Ar (g), N₂ (g) and air.

FIG. 2. Photoluminescence properties of the ZnS nanocrystals after being annealed under Ar (g), N₂ (g) and air.

FIG. 3. X-ray diffraction (XRD) patterns for precursor CuS nanoparticles, as produced by microbial fermentation, and XRD patterns for CuO nanoparticles produced after an 800° C. annealing step of the precursor nanoparticles in air at different temperature ramp rates.

FIG. 4. X-ray diffraction (XRD) patterns for precursor SnS nanoparticles, as produced by microbial fermentation, and XRD patterns for SnO₂ nanoparticles produced after an 800° C. annealing step of the precursor nanoparticles in air at different temperature ramp rates.

### DETAILED DESCRIPTION OF THE INVENTION

In the process described herein, non-oxide metal-containing particles (i.e., “non-oxide precursor particles”) are subjected to an oxidation step (typically air or liquid) that converts the non-oxide precursor particles to metal oxide particles. In particular embodiments, the oxidation step is conducted in an oxygen-containing atmosphere at a sufficiently elevated temperature to convert the non-oxide precursor particles to metal oxide particles. The oxygen-containing atmosphere is any atmosphere containing an effective level of oxygen gas to permit conversion of the precursor particles. The oxygen-containing atmosphere is commonly unmodified air (approximately 18-22% oxygen), but may also be elevated in oxygen (e.g., at least or above 22%, 25%, 30%, 35%, 40%, 45%, or 50% oxygen) or decreased in oxygen (e.g., up to or less than 15%, 10%, 5%, or 1% oxygen), and may also be in the form of an artificial gas mixture, such as an oxygen-nitrogen, oxygen-argon, oxygen-helium, or oxygen-carbon dioxide mixture. The oxygen-containing atmosphere may alternatively or in addition contain an oxidizing gas other than oxygen gas, such as ozone (O₃), nitrous oxide (N₂O), nitrogen dioxide (NO₂), and the halogen oxides (e.g., ClO₂). Generally, the oxidation step has the effect of volatizing chalcogen or pnictogen elements and replacing at least a portion or all of them with oxide atoms, wherein the one or more metal species in the precursor particles may or may not become oxidized to higher valence states. In other embodiments, the non-oxide precursor particles are oxidized by being coated (e.g., by spraying or dipping into a solution of the precursor particles) onto a substrate material, and then immersing the coated substrate into an oxidizing solution, such as a solution containing an inorganic or organic peroxide (e.g., H₂O₂ and urea peroxide), hypohalites (e.g., a hypochlorite salt, such as NaOCl), the halites (e.g., a chlorite or bromite salt, such as NaO₂Cl or NaO₂Br), the halates (e.g., a chlorate or bromate salt, such as NaO₃Cl or NaO₃Br), the perhalates (e.g., a perchlorate, perbromate, or periodate salt, such as NaO₄Cl, NaO₄Br, or NaO₄I), superoxides (e.g., NaO₂ and KO₂), ozone, pyrosulfates (e.g., NaS₂O₇), peroxodisulfates (e.g., Na₂S₂O₇, K₂S₂O₇, and (NH₄)₂S₂O₇), percarboxylic acids (e.g., peracetic acid), percarbonates, permanganates (e.g., K₂MnO₄) and/or an alkali hydroxide base (e.g., NaOH or KOH).

In some embodiments, the inventive method is practiced by treating the non-oxide precursor particles with an oxygen plasma. Any of the oxygen plasma processes known in the art, including high and low temperature plasma processes, are considered herein. The oxygen plasma can be, for example, a low temperature plasma (e.g., 15 to 30° C.) as commonly used in the art for surface modification and cleaning. Generally, the plasma process entails subjecting the precursor particles at reduced pressure (i.e., in a vacuum chamber) to a source of ionized oxygen or oxygen radicals. The ionized source of oxygen is typically produced by exposing oxygen at a reduced pressure of about 0.05 to 2 Torr to an ionizing source, such as an ionizing microwave, radiofrequency, or current source. Commonly, a radiofrequency source (e.g., of 13.56 MHz at a RF power of about 10-100 W) is used to ionize the oxygen. The particular oxygen plasma conditions depend on several factors including the type of plasma generator, gas composition, power source capability and characteristics, operating pressure and temperature, the degree of oxygenation required, and the composition of the precursor particles being treated (i.e., its susceptibility or resistance to oxidation). Depending on several factors including those mentioned above, the precursor particles may be exposed to the ionized oxygen for 0.1, 0.2, 0.5, 1, 1.5, 2, 5, 10, 12, 15, 20, 30, 40, 50, or 60 minutes. In other embodiments, a lower temperature (e.g., less than 15° C., or up to or less than 10, 5, or 0° C.) or a higher temperature (e.g., above, up to, or less than 30° C., such as above, up to, or less than 40° C., 50° C., 60° C., 70° C., 80° C., 90° C., 100° C., 150° C., 200° C., 250° C., 300° C., 350° C., or 400° C.) may be used. Generally, an oxygen plasma process is conducted as a combustionless process, i.e., without producing oxide gases of combustion.

In some embodiments, the non-oxide precursor particles are vapor-oxidized by a pulsed or non-pulsed thermal process. For example, the layer of precursor particles can be deposited on a substrate and oxidized by heating in a furnace under an oxygen-containing atmosphere, or by convecting heat through the substrate, such as by a hot plate, or by heating with a dispersed or focused (e.g., laser) form of high-energy electromagnetic radiation, such as infrared, ultraviolet, visible, microwave, x-ray, or radiowave forms of electromagnetic radiation, or by heating with a particle beam (e.g., electron or neutron beam), or with a plasma.

In different embodiments, the electromagnetic radiation used in the non-pulsed or pulsed thermal method can have a wavelength of precisely, about, at least, up to, or less than 0.1 nm, 1 nm, 10 nm, 50 nm, 100 nm, 200 nm, 250 nm, 300 nm, 350 nm, 400 nm, 450 nm, 500 nm, 550 nm, 600 nm, 650 nm, 700 nm, 750 nm, 800 nm, 850 nm, 900 nm, 950 nm, 1000 nm, 1050 nm, 1100 nm, 1200 nm, 1300 nm, 1400 nm, 1500 nm, 2000 nm, 2500 nm, 3000 nm, 4000 nm, 5000 nm, 8000 nm, 10 μm, 15 μm, 20 μm, 25 μm, 30 μm, 35 μm, 40 μm, 45 μm, 50 μm, 60 μm, 70 μm, 80 μm, 90 μm, 100 μm, 125 μm, 150 μm, 175 μm, 200 μm, 250 μm, 300 μm, 350 μm, 400 μm, 500 μm, 600 μm, 700 μm, 800 μm, 900 μm, 1 mm, mm, 10 mm, 25 mm, 50 mm, 100 mm, 500 mm, 1 cm, 5 cm, 10 cm, 100 cm, 500 cm, 1 m, 2 m, 5 m, 10 m, 50 m, 100 m, 500 m, or 1000 m, or a wavelength within a range bounded by any two of the foregoing exemplary wavelengths.

In particular embodiments, the invention is directed to a method of forming a film from a layer of particles by oxidizing (and possibly also melting or fusing) the layer of precursor particles with a pulse of thermal energy. In the method, a layer of particles (or a portion thereof), wherein the precursor particles typically have a size of up to or less than 100 microns, is oxidized by a pulse of thermal energy such that the precursor particles in the layer become oxidized, along with possible coalescence into a porous or non-porous planar form, if desired. Particles that coalesce lose their original shape by becoming substantially flattened, while also becoming connected, at least to some extent, with surrounding melted particles. In some embodiments, by suitable choice of particle composition, particle size, pulse power and pulse duration, the particles in the layer merge to form a continuous film (i.e., a film with no voids or pores). In other embodiments, by suitable choice of particle composition, particle size, pulse power and pulse duration, the particles in the layer merge to form a film that contains a degree of porosity.

The pulse thermal method considered herein can be any method that can subject a layer of particles to a pulse of intense thermal (i.e., radiant) energy. Generally, the means by which the radiant energy is produced does not substantially alter or degrade the composition of the particles. In particular embodiments, the radiant pulse is provided by an intense pulse of electromagnetic radiation. To produce heat in a material, the electromagnetic radiation is generally absorbed by the material and emitted as thermal energy.

The oxidation step can employ any temperature sufficient to oxidize (or possibly melt or fuse) the particles to be oxidized. The temperature of the oxidation step can widely vary depending on the composition of the particles and the type of oxide particles desired (e.g., crystalline vs. amorphous). In different embodiments, the oxidation step employs a temperature of precisely, about, at least, above, up to, or less than, for example, 50, 75, 100, 120, 125, 150, 200, 250, 300, 350, 400, 450, 500, 600, 700, 800, 900, 1000, 1500, 1800, 2000, 2200, 2500, or 3000 degrees Celsius (° C.), or a temperature within a range bounded by any two of the foregoing exemplary temperature values, wherein the term “about”, used for the temperature, generally indicates within ±5, ±4, ±3, ±2, or ±1° C. of the indicated temperature. In particular embodiments, the oxidation step is conducted at a low temperature, e.g., a temperature of −10, −5, 0, 5, 10, 15, 20, 25, 30, 35, or 40° C., or a temperature within a range bounded by any two of the foregoing temperatures, or a temperature within a range bounded by a minimum temperature of up to 40° C. (as above) and a maximum temperature of at least 50° C. (as above). In some embodiments, the process is conducted at room or ambient temperature, which is typically a temperature of 18-30° C., more typically 20-25° C., or about 22° C.

In some embodiments, a temperature ramping rate (gradient) is used to reach a final annealing temperature. The temperature ramping rate can be at precisely, about, at least, above, up to, or less than, for example, 1, 2, 5, 10, 20, 30, 40, 50, 60, 70, 80, 90, 100, 150, 200, 250, 300, 350, 400, 450, or 500° C./min, or a ramping rate within a range bounded by any two of the foregoing values. In particular embodiments, it has herein been discovered that the temperature ramping rate can have a pronounced effect on the size and composition of the resulting metal oxide nanoparticles. Thus, by judicious selection of the temperature ramping rate, metal oxide nanoparticles of a selected size and composition can be obtained.

In the pulsed version of the method, one or more pulses are applied to the layer of precursor particles (i.e., particles in the precursor layer) to achieve oxidation of the particles. In one embodiment, a single pulse achieves oxidation (along with possible melting and film formation) of the particles in the precursor layer. In another embodiment, more than one pulse (e.g., two, three, or a multiplicity of pulses), separated by a time interval between pulses, achieves oxidation (and possible melting and film formation) of the precursor particles. The pulse duration of each pulse can widely vary depending on such factors as the absorbing ability of the particles, the particle size, the wavelength of light, the temperature, and substrate (underlying layers) used. It is understood that a longer pulse duration generally results in a higher applied temperature.

Generally, the pulse duration is no more than 10, 5, or 1 second, and more typically, 100-500 milliseconds (ms). In different embodiments, the pulse duration can be precisely, about, at least, up to, or less than, for example, 1 second (i.e., 1000 ms), 500 ms, 400 ms, 300 ms, 200 ms, 100 ms, 50 ms, 20 ms, 10 ms, 5 ms, 1 ms (i.e., 1000 microseconds, i.e., 1000 μs, 900 μs, 800 μs, 700 μs, 600 μs, 500 μs, 400 μs, 300 μs, 200 μs, 100 μs, 80 μs, 50 μs, 40 μs, 30 μs, 20 μs, 10 μs, 5 μs, 2.5 μs, 1 μs, 0.5 μs, 0.25 μs, or 0.1 μs, or a pulse duration within a range bounded by any of the foregoing exemplary values. In different embodiments, the pulse energy can be, precisely, about, at least, up to, or less than, for example, 1, 2, 5, 10, 25, 50, 100, 200, 300, 400, 500, 600, 700, 800, 900, or 1000 J/cm². As known in the art, a pulse power (i.e., in W/cm²) can be derived by dividing the pulse energy (i.e., in J/cm²) by the pulse duration (in seconds). In the particular case of thermally treating precursor non-oxide particles on thermally sensitive substrates (e.g., a plastic), the pulse thermal process preferably employs a high energy density (e.g., >20 KW/cm²) thermal pulse at low ambient temperature.

If multiple pulses are used, the pulse duration may be the same or the pulse duration may vary across different pulses. For example, in different embodiments, the pulse duration alternates, or successively increases or decreases with time. When multiple pulses are used, the time interval between pulses (i.e., the periodicity) can also be appropriately selected. In different embodiments, the time interval is maintained below the pulse duration, maintained above the pulse duration, or increased or decreased with time successively or in a pattern-wise manner. The time interval can be, for example, precisely, about, at least, up to, or less than, for example, any of the exemplary values provided above for pulse duration, typically no more than about 1 or 2 seconds. The time interval may also be within a range bounded by any of the aforesaid values and/or any of the values provided above for pulse duration. The frequency of the pulses can be precisely, about, at least, up to, or less than, for example, 1 min⁻¹, 10 min⁻¹, 20 min⁻¹, 30 min⁻¹, 40 min⁻¹, 50 min⁻¹, 1 sec⁻¹ (1 Hz), 5 sec⁻¹, 10 sec⁻¹, 20 sec⁻¹, 30 sec⁻¹, 40 sec⁻¹, 50 sec⁻¹, 100 sec⁻¹, 500 sec⁻¹, 1000 sec⁻¹, 5000 sec⁻¹, 1×10⁴ sec⁻¹, 5×10⁴ sec⁻¹, 1×10⁵ sec⁻¹, 5×10⁵ sec⁻¹, 1×10⁶ sec⁻¹, 5×10⁶ sec⁻¹, 1×10⁷ sec⁻, or 5×10⁷ sec⁻¹, or a frequency within a range bounded by any of the foregoing exemplary values.

The pulse of electromagnetic radiation may be suitably adjusted in several other ways. For example, the pulse of electromagnetic radiation can be suitably adjusted, by means well known in the art, in its amplitude, phase, and extent of collimation. Collimation can be achieved by, for example, use of a collimator, such as a collimation lens or parabolic or spherical mirrors. Substantially collimated light corresponds to a laser emission, which is also considered herein as the pulse of electromagnetic radiation. The spectrum of the impinging radiation may also be appropriately filtered to provide particular wavelengths or a narrowed range of wavelengths.

The pulse of electromagnetic radiation can also be suitably adjusted in its power (i.e. intensity). The intensity of the pulse of electromagnetic radiation is generally at least 100 W/cm². In different embodiments, the pulse of electromagnetic radiation can be precisely, about, at least, or above, for example, 100, 200, 500, 1000, 2000, 3000, 4000, 5000, 6000, 7000, 8000, 9000, 1×10⁴, 1.5×10⁴, 2×10⁴, 2.5×10⁴, 3×10⁴, 3.5×10⁴, 4×10⁴, 4.5×10⁴, 5×10⁴, 5.5×10⁴, 6×10⁴, 6.5×10⁴, 7×10⁴, 7.5×10⁴, 8×10⁴, 9×10⁴, or 1×10⁵ W/cm², or an intensity within a range bounded by any of the foregoing exemplary values.

In particular embodiments, the pulsed thermal method employs a stabilized plasma arc high intensity radiation source, as described, for example, in U.S. Pat. Nos. 4,027,185 and 4,700,102, the contents of which are incorporated herein by reference in their entirety. As described in said patents, the arc can be suitably restricted by use of a vortexing liquid wall. Numerous modifications and improvements of the plasma arc method are known. For example, the instant invention incorporates by reference the contents of U.S. Pat. No. 4,937,490, which describes a high intensity radiation arc apparatus that includes liquid injecting means, gas injecting means, and exhausting means in the arc chamber in order to provide a liquid vortex motion and a gas vortex motion to restrict the plasma arc. Further adjustments, modifications, and optimizations of the processes and apparatuses taught in the foregoing patents can be made to better conform with the aims and goals of the instant invention, as described above. For example, the processes and apparatuses taught in the foregoing patents can be configured to emit a high intensity of electromagnetic radiation, particularly of the infrared wavelengths. Other modifications not contemplated in said foregoing patents may also be necessary to make the arc plasma systems described therein capable of operating within the parameters described herein, e.g., to provide any of the particular pulse durations, frequencies, power, or wavelengths described above. In particular embodiments, the thermal pulse method described herein utilizes a plasma arc lamp with an argon plasma. The use of a plasma arc lamp with an argon plasma provides the particular advantage of providing a significantly increased operating space compared to other thermal pulse configurations of the art, such as those using a flash lamp, particularly a xenon flash lamp.

Other methods for applying a thermal pulse are considered herein. For example, in some embodiments, a rapid physical heating process, such as by use of a heated resistor filament or other heated element in proximity to the layer of precursor particles, can be utilized. In such a heating process, a capacitor may be employed for storing and releasing a large amount of electrical energy to the heating element, thereby generating a quick pulse of thermal energy. In other embodiments, a pulse of direct or alternating current may be applied to the substrate. By appropriate selection of such characteristics as current level, amplitude, and frequency, the pulse of current can be adjusted to melt at least a portion of the layer of precursor particles. The frequency of the alternating current can be any suitable frequency, particularly a radiofrequency. In some embodiments, one or more of any of the means, described above, for generating a thermal pulse is excluded from the method described herein. In still other embodiments, a combination of any of the heating means described above is used in the film-forming method described herein.

The non-oxide precursor particles can have any non-metal oxide composition known in the art that can be converted to an oxide form by an oxidation process. The non-oxide precursor particles contain at least one chalcophile metal and at least one non-oxide main group element, typically at least one chalcogen element in a negative oxidation state, i.e., sulfur (S), selenium (Se), and tellurium (Te), and/or at least one pnictogen element in a negative oxidation state, i.e., nitrogen (N), phosphorus (P), arsenic (As), and bismuth (Bi). The chalcophile metal is one, as known in the art, which has a propensity for forming metal-chalcogenide (i.e., metal-sulfide, metal-selenide, and metal-telluride) compositions. Some examples of chalcophile metals include, for example, Sc, Ti, V, Cr, Mn, Fe, Co, Ni, Cu, Zn, Y, Zr, Nb, Hf, Ta, Cd, Mo, W, Ag, Pd, Pt, Ga, In, Tl, Ge, Sn, Pb, Sb, and Bi. Some metals particularly considered herein include Cd, Cu, Fe, Ga, In, Sn, and Zn.

In some embodiments, the non-oxide precursor particles have a mono-metal or mixed-metal chalcogenide or pnictide composition of the general formula:

[(M′ₓM″_{w-v})^{+r}]ₛX^{-m}_{z} (1)

In Formula (1) above, each of M′ and M″ represents at least one metal cation, at least one of which is a transition metal cation, wherein M′ and M″ are the same or different, X represents S, Se, Te, N, P, As, Sb, or Bi, or a combination thereof, and the subscripts w, v, x, s, r, m, and z are integer or fractional numbers that together maintain charge balancing, wherein r×s=m×z, wherein “x” in the latter equation represents multiplication, unlike “x” in Formula (1) where it represents a variable. M′ and M″ can independently be any of the metal cations described above. Some examples of such compositions, which can be considered quantum dot compositions, include CdS, CdSe, CdTe, CdSₓSe₁₋ₓ, Cd₃As₂, ZnS, ZnSe, ZnTe, ZnSₓSe₁₋ₓ, Zn₃As₂, Ga₂S₃, Ga₂Se₃, Ga₂Te₃, GaAs, In₂S₃, In₂Se₃, In₂Te₃, InAs, CuS, CuSe, CuTe, Cu₃As₂, FeSe, Fe₃As₂, FeAs, PbS, PbSe, PbTe, Pb₃As₂, HgS, HgSe, HgTe, CdₓZn₁₋ₓTe, CdₓHg₁₋ₓTe, HgₓZn₁₋ₓTe, CdₓZn₁₋ₓS, CdₓZn₁₋ₓSe, CdₓZn₁₋ₓS_{y}Se_{1-y}, CdₓHg₁₋ₓSe, HgₓZn₁₋ₓSe, PbₓSn₁₋ₓTe, GaₓIn₂₋ₓSe₃, and GaₓIn₁₋ₓAs, wherein x and y are, independently, an integral or non-integral numerical value greater than 0 and less than or equal to 1 (or less than or equal to 2 for the expression 2-x).

In other embodiments, the non-oxide precursor particles have a composition encompassed by the following general formula:

Cu(InₓGa₁₋ₓ)X′₂ (2)

In Formula (2) above, x is an integral or non-integral numerical value of or greater than 0 and less than or equal to 1, and X′ represents at least one non-metal selected from S, Se, and Te. In different embodiments, X′ represents S, Se, Te, or a combination of two or three of these elements. X′ can also be represented by the formula SⱼSeₖTeₘ, wherein j, k, and m are independently 0 or an integral or non-integral numerical value greater than 0 and less than or equal to 1, provided that the sum of j, k, and m is 1. Compositions according to Formula (2) and subformulas encompassed therein are collectively referred to herein as CIGs compositions. The CIGs compositions encompassed by Formula (2) may also contain a relative molar ratio of Cu that diverges from 1.

In particular embodiments, the CIGs composition is according to the following sub-formula:

CuInₓGa₁₋ₓ)S₂ (2a)

Some specific examples of compositions according to Formula (2a) include CuInS₂, CuIn_{0.9}Ga_{0.1}S₂, CuIn_{0.8}Ga_{0.2}S₂, CuIn_{0.7}Ga_{0.3}S₂, CuIn_{0.6}Ga_{0.4}S₂, CuIn_{0.5}Ga_{0.5}S₂, CuIn_{0.4}Ga_{0.6}S₂, CuIn_{0.3}Ga_{0.7}S₂, CuIn_{0.2}Ga_{0.8}S₂, CuIn_{0.1}Ga_{0.9}S₂, and CuGaS₂.

In other particular embodiments, the CIGs composition is according to the following sub-formula:

CuInₓGa₁₋ₓSe₂ (2b)

Some specific examples of compositions according to Formula (2b) include CuInSe₂, CuIn_{0.9}Ga_{0.1}Se₂, CuIn_{0.8}Ga_{0.2}Se₂, CuIn_{0.7}Ga_{0.3}Se₂, CuIn_{0.6}Ga_{0.4}Se₂, CuIn_{0.5}Ga_{0.5}Se₂, CuIn_{0.4}Ga_{0.6}Se₂, CuIn_{0.3}Ga_{0.7}Se₂, CuIn_{0.2}Ga_{0.8}Se₂, CuIn_{0.1}Ga_{0.9}Se₂, and CuGaSe₂.

In yet other particular embodiments, the CIGs composition is according to the following sub-formula:

CuInₓGa₁₋ₓTe₂ (2c)

Some specific examples of compositions according to Formula (2c) include CuInTe₂, CuIn_{0.9}Ga_{0.1}Te₂, CuIn_{0.8}Ga_{0.2}Te₂, CuIn_{0.7}Ga_{0.3}Te₂, CuIn_{0.6}Ga_{0.4}Te₂, CuIn_{0.5}Ga_{0.5}Te₂, CuIn_{0.4}Ga_{0.6}Te₂, CuIn_{0.3}Ga_{0.7}Te₂, CuIn_{0.2}Ga_{0.8}Te₂, CuIn_{0.1}Ga_{0.9}Te₂, and CuGaTe₂.

In some embodiments, the non-oxide precursor particles have a composition encompassed by the following general formula:

MₓX″X′_{y} (3)

In Formula (3) above, M represents at least one chalcophile (for example, divalent or monovalent) metal species other than Sn, X″ is selected from Ge, Sn, As, and Sb, or a combination thereof, X′ is selected from S, Se, and Te, x is 2 or 3, and y is 2, 3, or 4 (more typically, 3 or 4). In particular embodiments, M represents one, two, three, or four metals selected from Cu, Fe, Zn, and Cd.

In particular embodiments of Formula (3), the non-oxide precursor particles have a quaternary kesterite-type composition encompassed by the following general formula:

M₃SnX′₄ (4)

In Formula (4) above, M represents at least one chalcophile metal other than Sn, and X′ is as defined above. The relative molar ratio of Sn encompassed by Formula (4) may diverge from 1.

In some embodiments, the kesterite-type compositions of Formula (4) are encompassed by the following sub-generic formula:

Cu₃₋ₓM′ₓSnX′₄ (4a)

In Formula (4a), M′ represents one or more chalcophile metals other than Cu, and X′ is as defined above (S, Se, and/or Te). In particular embodiments, M′ represents one, two, or three metals selected from any chalcophile metal, such as, for example, V, Cr, Mn, Co, Ni, Fe, Zn, Cd, Cu, Mo, W, Pd, Pt, Au, Ag, Hg, Ga, In, Tl, Ge, Sn, Pb, Sb, and Bi. Some metals particularly considered herein include Fe, Zn, and Cd. The subscript x is an integral or non-integral numerical value of or greater than 0 and up to or less than 1, 2, or 3. In different embodiments, x can be selected to be a value of precisely or about 1, 2, or 3, or a non-integral value between 0 and 3, wherein the term “about” generally indicates within ±0.5, ±0.4, ±0.3, ±0.2, or ±0.1 of the value. For example, a value of about 1 generically indicates, in its broadest sense, that x can be 0.5 to 1.5 (i.e., 1±0.5).

Some particular kesterite-type compositions of Formula (4a) are encompassed by the following sub-generic formula:

Cu₃₋ₓZnₓSnX′₄ (4a-1)

In Formula (4a-1), x and X′ are as described above under Formula (3) or (4a). Some specific examples of compositions according to Formula (4a-1) when X′ is S include Cu₃SnS₄ (kuramite), Cu₂ZnSnS₄ (kesterite), CuZn₂SnS₄, Cu_{0.5}Zn_{2.5}SnS₄, Cu_{2.5}Zn_{0.5}SnS₄, Cu_{1.5}Zn_{1.5}SnS₄, and Zn₃SnS₄. Other examples of compositions according to Formula (4a-1) are provided by replacing S in the foregoing examples with Se, Te, or a combination of non-metals selected from S, Se, and Te. The relative molar ratio of Sn encompassed by formula (4a-1) may diverge from 1.

Other particular kesterite-type compositions of formula (4a) are encompassed by the following sub-generic formula:

Cu₃₋ₓFeₓSnX′₄ (4a-2)

In Formula (4a-2), x and X′ are as described above under Formula (4a). Some specific examples of compositions according to Formula (4a-2) when X is S include Cu₃SnS₄, Cu₂FeSnS₄ (stannite), CuFe₂SnS₄, Cu_{0.5}Fe_{2.5}SnS₄, Cu_{2.5}Fe_{0.5}SnS₄, Cu_{1.5}Fe_{1.5}SnS₄, and Fe₃SnS₄. Other examples of compositions according to Formula (4a-2) are provided by replacing S in the foregoing examples with Se, Te, or a combination of non-metals selected from S, Se, and Te. The relative molar ratio of Sn encompassed by Formula (4a-2) may diverge from 1.

Other particular kesterite-type compositions of Formula (4a) are encompassed by the following sub-generic formula:

Cu₃₋ₓCdₓSnX′₄ (4a-3)

In Formula (4a-3), x and X′ are as described above under Formula (4a). Some specific examples of compositions according to Formula (4a-3) when X is S include Cu₃SnS₄, Cu₂CdSnS₄ (cernyite), CuCd₂SnS₄, Cu_{0.5}Cd_{2.5}SnS₄, Cu_{2.5}Cd_{0.5}SnS₄, Cu_{1.5}Cd_{1.5}SnS₄, and Cd₃SnS₄. Other examples of compositions according to Formula (4a-3) are provided by replacing S in the foregoing examples with Se, Te, or a combination of non-metals selected from S, Se, and Te. The relative molar ratio of Sn encompassed by Formula (4a-3) may diverge from 1.

In other embodiments, the kesterite-type compositions of Formula (4) are encompassed by the following sub-generic formula:

Cu₂M′ₓM′₁₋ₓSnX′₄ (4b)

In Formula (4b), each M′ is defined as above under Formula (4a), x is an integral or non-integral numerical value of or greater than 0 and up to or less than 1, and X′ is as defined above. In particular embodiments, the two M′ metals in Formula (4b) are not the same, i.e., the two M′ metals in Formula (4b) are different. The relative molar ratio of Sn encompassed by Formula (4b) may diverge from 1, and the relative molar ratio of Cu encompassed by Formula (4b) may diverge from 2.

Some particular kesterite-type compositions of Formula (4b) are encompassed by the following sub-generic formula:

Cu₂FeₓZn₁₋ₓSnX′₄ (4b-1)

Some specific examples of compositions according to Formula (4b-1) when X is S include Cu₂Fe_{0.1}Zn_{0.9}SnS₄, Cu₂Fe_{0.2}Zn_{0.8}SnS₄, Cu₂Fe_{0.3}Zn_{0.3}SnS₄, Cu₂Fe_{0.4}Zn_{0.6}SnS₄, Cu₂Fe_{0.5}Zn_{0.5}SnS₄, Cu₂Fe_{0.6}Zn_{0.4}SnS₄, Cu₂Fe_{0.7}Zn_{0.3}SnS₄, Cu₂Fe_{0.8}Zn_{0.2}SnS₄, and Cu₂Fe_{0.9}Zn_{0.1}SnS₄. Other examples of compositions according to Formula (4b-1) are provided by replacing S in the foregoing examples with Se, Te, or a combination of non-metals selected from S, Se, and Te. The relative molar ratio of Sn encompassed by Formula (4b-1) may diverge from 1, and the relative molar ratio of Cu encompassed by Formula (4b-1) may diverge from 2.

In other embodiments, the kesterite-type compositions of Formula (4) are encompassed by the following sub-generic formula:

CuM′ₓM′₂₋ₓSnX′₄ (4c)

In Formula (4c), each M′ is defined as above under Formula (4a), x is an integral or non-integral numerical value of at least or greater than 0 and up to or less than 1 or 2, and X′ is as defined above. In particular embodiments, the two M′ metals in Formula (4c) are not the same, i.e., the two M′ metals in Formula (4c) are different. In different embodiments, x can be selected to be a value of precisely or about 1 or 2, or a non-integral value between 0 and 2, wherein the term “about” is as defined under Formula (4a). The relative molar ratio of Sn and Cu encompassed by Formula (4c) may each diverge from 1.

Some particular kesterite-type compositions of Formula (4c) are encompassed by the following sub-generic formula:

CuFeₓZn₂₋ₓSnX′₄ (4c-1)

Some specific examples of compositions according to Formula (4c-1) when X′ is S (i.e., CuFeₓZn₂₋ₓSnS₄) include CuFe_{0.5}Zn_{1.5}SnS₄, CuFeZnSnS₄, and CuFe_{1.5}Zn_{0.5}SnS₄. Other examples of compositions according to Formula (4c-1) are provided by replacing S in the foregoing examples with Se, Te, or a combination of non-metals selected from S, Se, and Te. The relative molar ratio of Sn and Cu encompassed by Formula (4c-1) may each diverge from 1.

In other embodiments of Formula (3), the non-oxide precursor particles have a tertiary kesterite-type composition encompassed by the following general formula:

M₂SnX′₃ (5)

In Formula (5) above, M represents at least one chalcophile (typically divalent) metal other than Sn, as further described above, and X′ is as defined above. In particular embodiments, M represents one, two, three, or four metals selected from Cu, Fe, Zn, and Cd. The relative molar ratio of Sn encompassed by Formula (5) may diverge from 1. Some examples of compositions according to Formula (5) include Cu₂SnS₃, Cu₂SnSe₃, Cu₂SnTe₃, Fe₂SnS₃, Fe₂SnSe₃, Fe₂SnTe₃, Zn₂SnS₃, Zn₂SnSe₃, Zn₂SnTe₃, Cd₂SnS₃, Cd₂SnSe₃, and Cd₂SnTe₃, as well as such composition wherein X′ includes a combination of two or three chalcogens selected from S, Se, and Te, e.g., Cu₂SnSSe₂, and/or wherein M represents two or more metal species, e.g., CuZnSnS₃, CuCdSnS₃, CuFeSnS₃, ZnCdSnS₃, CuZnSnSe₃, and CuZnSnTe₃.

In other embodiments of Formula (3), the non-oxide precursor particles have a thermoelectric composition encompassed by the following general formula:

M₃SbX′₄ (6)

In Formula (6) above, M represents at least one chalcophile (typically divalent) metal other than Sb, as further described above, and X′ is as defined above. In particular embodiments, M represents one, two, three, or four metals selected from Cu, Fe, Zn, and Cd. The relative molar ratio of Sb encompassed by Formula (6) may diverge from 1. Some examples of compositions according to Formula (6) include Cu₃SbS₄, Cu₃SbSe₄, Cu₃SbTe₄, Fe₃SbS₄, Fe₃SbSe₄, Fe₃SbTe₄, Zn₃SbS₄, Zn₃SbSe₄, Zn₃SbTe₄, Cd₃SbS₄, Cd₃SbSe₄, and Cd₃SbTe₄, as well as such composition wherein X′ includes a combination of two or three chalcogens selected from S, Se, and Te, e.g., Cu₃SbSSe₃, and/or wherein M represents two or more metal species, e.g., Cu₂ZnSbS₃, Cu₂CdSbS₃, Cu₂FeSbS₃, ZnCdSbS₃, Cu₂ZnSbSe₃, and Cu₂ZnSbTe₃.

In other embodiments of Formula (3), the non-oxide precursor particles have a thermoelectric composition encompassed by the following general formula:

M₃GeX′₄ (7)

In Formula (7) above, M represents at least one chalcophile (typically divalent) metal other than Ge, as further described above, and X′ is as defined above. In particular embodiments, M represents one, two, three, or four metals selected from Cu, Fe, Zn, and Cd. The relative molar ratio of Ge encompassed by Formula (7) may diverge from 1. Some examples of compositions according to Formula (7) include Cu₃GeS₄, Cu₃GeSe₄, Cu₃GeTe₄, Fe₃GeS₄, Fe₃GeSe₄, Fe₃GeTe₄, Zn₃GeS₄, Zn₃GeSe₄, Zn₃GeTe₄, Cd₃GeS₄, Cd₃GeSe₄, and Cd₃GeTe₄, as well as such composition wherein X′ includes a combination of two or three chalcogens selected from S, Se, and Te, e.g., Cu₃GeSSe₃, and/or wherein M represents two or more metal species, e.g., Cu₂ZnGeS₃, Cu₂CdGeS₃, Cu₂FeGeS₃, ZnCdGeS₃, Cu₂ZnGeSe₃, and Cu₂ZnGeTe₃.

In other embodiments of Formula (3), the non-oxide precursor particles have a thermoelectric composition encompassed by the following general formula:

M₃ArX′₄ (8)

In Formula (8) above, M represents at least one chalcophile (typically divalent) metal other than Ar, as further described above, and X′ is as defined above. In particular embodiments, M represents one, two, three, or four metals selected from Cu, Fe, Zn, and Cd. The relative molar ratio of Ar encompassed by Formula (8) may diverge from 1. Some examples of compositions according to Formula (8) include Cu₃ArS₄, Cu₃ArSe₄, Cu₃ArTe₄, Fe₃ArS₄, Fe₃ArSe₄, Fe₃ArTe₄, Zn₃ArS₄, Zn₃ArSe₄, Zn₃ArTe₄, Cd₃ArS₄, Cd₃ArSe₄, and Cd₃ArTe₄, as well as such composition wherein X′ includes a combination of two or three chalcogens selected from S, Se, and Te, e.g., Cu₃ArSSe₃, and/or wherein M represents two or more metal species, e.g., Cu₂ZnArS₃, Cu₂CdArS₃, Cu₂FeArS₃, ZnCdArS₃, Cu₂ZnArSe₃, and Cu₂ZnArTe₃.

As used herein, the term “metal oxide” indicates compounds or materials containing at least one metal species and oxide atoms, and the term “mixed-metal oxide” indicates compounds or materials containing at least two different metal species and oxide atoms. When more than one metal is included, the metals may be substantially intermixed throughout the mixed-metal oxide such that separate phases do not exist. Alternatively, the different metals may form distinct phases composed of different metal oxide compositions in the mixed-metal oxide. The metal oxide compounds or materials may or may not further contain, for example, one or more dopant or trace metal species, chemisorbed water, water of hydration, or adsorbed molecular groups. Generally, the oxide composition is derived from the non-oxide precursor compositions by replacing at least a portion or all chalcogen or pnictogen species therein with oxide atoms. In other embodiments, the oxide composition not only replaces a portion or all of the chalcogen or pnictogen species in the precursor composition, but also changes the stoichiometric relationship between elements in the composition.

In a first set of embodiments, the produced metal oxide particles have an oxide composition that contains one metal species, which is herein designated as a mono-metal oxide composition. In a second set of embodiments, the produced metal oxide particles have an oxide composition that contains at least two (or at least three, four, or more) metal species, which is herein designated as a mixed-metal oxide composition. In particular embodiments, the metal oxide composition correspond to any of the metal chalcogenide or metal pnictide compositions provided above, except that the chalcogenide or pnictide species (generalized as X) is at least partially or completely replaced with oxide (O).

In some embodiments, the one or more metal species in the metal oxide composition is or includes a transition metal, i.e., Groups III-XII (scandium through zinc groups) of the Periodic Table. In some embodiments, the metal species is or includes a first-row transition metal. Some examples of first-row transition metal ions include Sc(III), Ti(IV), V(III), V(IV), V(V), Cr(III), Cr(VI), Mn(VII), Mn(V), Mn(IV), Mn(III), Fe(II), Fe(III), Co(III), Ni(III), Cu(I), and Cu(II). In other embodiments, the metal species is or includes a second-row transition metal. Some examples of second-row transition metal ions include Y(III), Zr(IV), Nb(IV), Nb(V), Mo(IV), Mo(VI), Ru(IV), Ru(VIII), Rh(III), Rh(IV), Pd(II), Ag(I), and Cd(II). In other embodiments, the metal species is or includes a third-row transition metal. Some examples of third-row transition metal species include Hf(IV), Ta(V), W(III), W(IV), W(VI), Re(IV), Re(VII), Ir(IV), Pt(IV), and Au(III). Some examples of metal oxide compositions containing a transition metal include the mono-metal oxide compositions Sc₂O₃, TiO₂, Cr₂O₃, Fe₂O₃, Fe₃O₄, FeO, Co₂O₃, Ni₂O₃, CuO, Cu₂O, ZnO, Y₂O₃, ZrO₂, NbO₂, Nb₂O₅, RuO₂, PdO, Ag₂O, CdO, HfO₂, Ta₂O₅, WO₂, and PtO₂, as well as mixed-metal oxide compositions wherein one or more metals replace a portion of any of the metals in the foregoing compositions, e.g., replacing a portion of Fe in Fe₃O₄ with Co to result in CoFe₂O₄, or wherein any of the foregoing metal oxide compositions are in admixture. Other examples of metal oxide compositions include the paratungstates and polyoxometallates, e.g., polyoxomolybdates, polyoxotungstates, and polyoxovanadates.

In other embodiments, the one or more metal species in the metal oxide composition is or includes an alkali, alkaline earth, main group, or lanthanide metal. Some examples of alkali metal species include Li⁺, Na⁺, K⁺, and Rb⁺, which may be incorporated in such mono-metal oxide compositions as Li₂O, Na₂O, K₂O, and Rb₂O, Some examples of alkaline earth metal species include Be²⁺, Mg²⁺, Ca²⁺, and Sr²⁺, which may be incorporated in such mono-metal oxide compositions as BeO, MgO, CaO, and SrO. Some examples of main group metal species (e.g., cations of Group IIIA-VIIA of the Periodic Table), include B³⁺, Al³⁺, Ga³⁺, In³⁺, T¹⁺, Tl³⁺, Si⁺, Ge⁴⁺, Sn²⁺, Sn⁴⁺, Pb²⁺, Pb⁴⁺, N³⁺, P³⁺, P⁵⁺, As³⁺, As⁵⁺, Sb³⁺, Sb⁵⁺, and Bi³⁺, which may be incorporated in such mono-metal oxide composition as B₂O₃, Ga₂O₃, SnO, SnO₂, PbO, PbO₂, Sb₂O₃, Sb₂O₅, and Bi₂O₃. Some examples of lanthanide metal species include any of the elements in the Periodic Table having an atomic number of 57 to 71, e.g., La³⁺, Ce³⁺, Ce⁴⁺, Pr³⁺, Nd³⁺, Sm³⁺, Eu³⁺, Gd³⁺, and Tb³⁺, which may be incorporated in such mono-metal oxide composition as La₂O₃, Ce₂O₃, and CeO₂.

In a first set of embodiments, the produced metal oxide particles have an oxide composition that is a mono-metal oxide composition in which the metal species is selected from Sc, Ti, V, Cr, Mn, Fe, Co, Ni, Cu, Zn, Y, Zr, Nb, Hf, Ta, Cd, Mo, W, Ag, Pd, Pt, Ga, In, Tl, Ge, Sn, Pb, Sb, and Bi. In a second set of embodiments, the produced metal oxide particles have an oxide composition that is a mixed-metal oxide composition that includes at least one, two, three, or four metals selected from Sc, Ti, V, Cr, Mn, Fe, Co, Ni, Cu, Zn, Y, Zr, Nb, Hf, Ta, Cd, Mo, W, Ag, Pd, Pt, Ga, In, Tl, Ge, Sn, Pb, Sb, and Bi, or in which all of the metals are exclusively selected from the foregoing list of metal species.

In some embodiments, any one or more classes or specific types of metal species described above are excluded from the oxide composition. In other embodiments, two or more classes or specific types of metal species described above may be combined.

The metal oxide composition may or may not also include chalcogen or pnictogen elements remaining from the precursor, depending on the extent of oxidation achieved during the oxidation step. Thus, the metal oxide composition may or may not include such chalcogen or pnictogen elements as S, Se, Te, N, P, As, and/or Sb, wherein the chalcogen or pnictogen element may or may not be in an oxidized state, e.g., as sulfite (SO₃²⁻), sulfate (SO₄²⁻), selenite (SeO₃²⁻), selenate (SeO₄²⁻), tellurite (TeO₃²⁻), tellurate (TeO₄²⁻), nitrite (NO₂⁻), nitrate (NO₃⁻), phosphite (PO₃³⁻), phosphate (PO₄³⁻), arsenite (AsO₃³⁻), arsenate (AsO₄³⁻), antimonite (SbO₃³⁻), and antimonate (SbO₄³⁻).

In some embodiments, the metal oxide particles have a mono-metal or mixed-metal oxide composition of the general formula:

[(M′ₓM″_{w-x})^{+r}]ₛO_{y} (9)

In Formula (9) above, each of M′ and M″ represents at least one metal cation, at least one of which is a transition metal cation, wherein M′ and M″ are the same or different, and the subscripts w, x, s, r, and y are integer or fractional numbers that together maintain charge balancing, wherein r×s=2y. M′ and M″ can independently be any of the metal cations described above. Some examples of such compositions (e.g., CoFe₂O₄) have been provided above.

In some embodiments, the metal oxide particles have a perovskite structure of the formula:

M′M″O₃ (10)

In Formula (10) above, M′ and M″ are typically different metal cations, thereby being further exemplary of mixed-metal oxide compositions. The metal cations can be independently selected from, for example, the first, second, and third row transition metals, lanthanide metals, and main group (particularly Groups IIIA and IVA) metals, such as Pb and Bi. More typically, M′ represents a trivalent metal (often from Group IIIB) and M″ represents a transition metal, and more typically, a first row transition metal. Some examples of perovskite oxides include LaCrO₃, LaMnO₃, LaFeO₃, YCrO₃, and YMnO₃.

It is also possible for M′ and M″ in Formula (10) to be the same metal, wherein Formula (10) reduces to M′₂O₃. In these compositions, M′ is typically a first row transition metal. Some examples of such compositions include Cr₂O₃, and Fe₂O₃, both having the corundum crystal structure, and Mn₂O₃, having the bixbyite crystal structure.

In other embodiments, the metal oxide particles have a spinel structure of the formula:

Mₓ′M″₃₋ₓO₄ (11)

In Formula (11) above, M′ and M″ are the same or different metal cations. Typically, at least one of M′ and M″ is a transition metal cation, and more typically, a first-row transition metal cation. In order to maintain charge neutrality with the four oxide atoms, the oxidation states of M′ and M″ sum to +8. Generally, two-thirds of the metal ions are in the +3 state while one-third of the metal ions are in the +2 state. The +3 metal ions generally occupy an equal number of tetrahedral and octahedral sites, whereas the +2 metal ions generally occupy half of the octahedral sites. However, Formula (11) includes other chemically-acceptable possibilities, including that the +3 metal ions or +2 metal ions occupy only octahedral or tetrahedral sites, or occupy one type of site more than another type of site. The subscript x can be any numerical (integral or non-integral) positive value, typically at least 0.01 and up to 1.5.

When M′ and M″ in Formula (11) are the same, Formula (11) becomes simplified to the general formula:

M₃O₄ (12)

Some examples of compositions according to Formula (12) include Fe₃O₄ (magnetite), Co₃O₄, and Mn₃O₄.

Some examples of spinel oxide compositions having two metals include those of the general composition M′_{y}Fe_{3-y}O₄ (e.g., Ti_{y}Fe_{3-y}O₄, V_{y}Fe_{3-y}O₄, Cr_{y}Fe_{3-y}O₄, Mn_{y}Fe_{3-y}O₄, CO_{y}Fe_{3-y}O₄, Ni_{y}Fe_{3-y}O₄, CU_{y}Fe_{3-y}O₄, Zn_{y}Fe_{3-y}O₄, Pd_{y}Fe_{3-y}O₄, Pt_{y}Fe_{3-y}O₄, Cd_{y}Fe_{3-y}O₄, Ru_{y}Fe_{3-y}O₄, Zr_{y}Fe_{3-y}O₄, Nb_{y}Fe_{3-y}O₄, Gd_{y}Fe_{3-y}O₄, Eu_{y}Fe_{3-y}O₄, Tb_{y}Fe_{3-y}O₄, and Ce_{y}Fe_{3-y}O₄); the general composition M′_{y}Co_{3-y}O₄ (e.g., Ti_{y}Co_{3-y}O₄, V_{y}Co_{3-y}O₄, Cr_{y}Co_{3-y}O₄, Mn_{y}Co_{3-y}O₄, Ni_{y}Co_{3-y}O₄, Cu_{y}Co_{3-y}O₄, Zn_{y}Co_{3-y}O₄, Pd_{y}Co_{3-y}O₄, Pt_{y}Co_{3-y}O₄, Cd_{y}Co_{3-y}O₄, Ru_{y}Co_{3-y}O₄, Zr_{y}Co_{3-y}O₄, Nb_{y}Co_{3-y}O₄, Gd_{y}Co_{3-y}O₄, Eu_{y}Co_{3-y}O₄, Tb_{y}Co_{3-y}O₄, and Ce_{y}Co_{3-y}O₄); and the general composition M′_{y}Ni_{3-y}O₄ (e.g., TiNi_{3-y}O₄, V_{y}Ni_{3-y}O₄, Cr_{y}Ni_{3-y}O₄, Mn_{y}Ni_{3-y}O₄, Fe_{y}Ni_{3-y}O₄, CU_{y}Ni_{3-y}O₄, Zn_{y}Ni_{3-y}O₄, Pd_{y}Ni_{3-y}O₄, Pt_{y}Ni_{3-y}O₄, Cd_{y}Ni_{3-y}O₄, RU_{y}Ni_{3-y}O₄, Zr_{y}Ni_{3-y}O₄, Nb_{y}Ni_{3-y}O₄, Gd_{y}Ni_{3-y}O₄, Eu_{y}Ni_{3-y}O₄, Tb_{y}Ni_{3-y}O₄, and Ce_{y}Ni_{3-y}O₄), wherein y in the general compositions given above represents an integral or non-integral numerical value of at least 0.1 and up to 2; and M′ represents one or a combination of metal ions, e.g., (M′ₐ,M″_{b})_{y}Fe_{3-y}O₄, wherein subscripts a and b are non-integral numbers that sum to 1 (e.g., Mn_{0.5}Zn_{0.5}Fe₂O₄, Mn_{0.4}Zn_{0.6}Fe₂O₄, Ni_{0.5}Co_{0.5}Fe₂O₄, and Ni_{0.4}Co_{0.6}Fe₂O₄).

In particular embodiments of Formula (11), the spinel structure has the composition:

M′M″₂O₄ (13)

In Formula (13) above, M″ is typically a trivalent metal ion and M′ is typically a divalent metal ion. More typically, M′ and M″ independently represent transition metals, and more typically, first row transition metals. Some examples of spinel compositions include NiCr₂O₄, CuCr₂O₄, ZnCr₂O₄, CdCr₂O₄, MnCr₂O₄, NiMn₂O₄, CuMn₂O₄, ZnMn₂O₄, CdMn₂O₄, NiCo₂O₄, CuCo₂O₄, ZnCo₂O₄, CdCo₂O₄, MnCo₂O₄, NiFe₂O₄, CuFe₂O₄, ZnFe₂O₄, CdFe₂O₄, and MnFe₂O₄. M′ and M″ can also be combinations of metals, such as in (Co,Zn)Cr₂O₄, and Ni(Cr, Fe)₂O₄.

The metal oxide particles (and/or non-oxide precursor particles) can have any suitable particle size. The term “particle size”, as used herein, refers to the length of at least one, two, or all of the dimensions of the particle. In the specific case of symmetric particles (e.g., spherical. spheroidal, or polyhedral shapes), the particle size corresponds to the diameter of the particles. The metal oxide particles generally possess a particle size of up to 10 microns. In some embodiments, the metal oxide particles have a size in the nanoscale regime, i.e., less than 1 micron (1 μm). In different embodiments, the metal oxide particles have a size of precisely, about, at least, above, up to, or less than, for example, 1 nm, 2 nm, 3 nm, 4 nm, 5 nm, 10 nm, 12 nm, 15 nm, 20 nm, 25 nm, 30 nm, 40 nm, 50 nm, 100 nm, 150 nm, 200 nm, 250 nm, 300 nm, 400 nm, 500 nm, 600 nm, 700 nm, 800 nm, 900 nm, 1 μm, 2 μm, 5 μm, or 10 μm, or a size within a range bounded by any two of the foregoing exemplary particle sizes (e.g., 1-10 nm, 2-10 nm, 1-20 nm, 2-20 nm, 3-20 nm, 1-50 nm, 2-50 nm, 5-50 nm, 10-50 nm, 1-100 nm, 5-100 nm, 10-100 nm, 20-100 nm, 1-500 nm, 10-500 nm, 50-500 nm, 1-1000 nm, 10-1000 nm, or 50-1000 nm). In some embodiments, the particles are fairly disperse in size (e.g., having a size variation of 20%, 30%, 40%, 50%, or greater from a median or mean size). In other embodiments, the particles are fairly monodisperse in size (e.g., having a size variation of or less than 50%, 40%, 30%, 20%, 10%, 5%, 2%, or 1% from a median or mean size).

The metal oxide particles (and/or non-oxide precursor particles) can also have any suitable morphology. Some examples of possible particle shapes include amorphous, fibrous, tubular, cylindrical, rod, needle, spherical, ovoidal, pyramidal, cuboidal, rectangular, dodecahedral, octahedral, plate, and tetrahedral. In some embodiments, the metal oxide particles are equiaxed euhedral crystals (i.e., typically cubes, octahedra, and modifications thereof).

In some embodiments, the metal oxide particles produced by the methodology described herein possess at least one photoluminescence absorption or emission peak. The peak can be, for example, in the UV, visible, and/or IR range. In different embodiments, the photoluminescence peak is located at, or at least, or above, or less than 200 nm, 250 nm, 300 nm, 320 nm, 340 nm, 360 nm, 380 nm, 400 nm, 420 nm, 440 nm, 460 nm, 480 nm, 500 nm, 520 nm, 540 nm, 560 nm, 580 nm, 600 nm, 620 nm, 640 nm, 660 nm, 680 nm, 700 nm, 720 nm, 740 nm, 760 nm, 780 nm, 800 nm, 820 nm, 840 nm, 860 nm, 880 nm, 900 nm, 920 nm, 940 nm, 960 nm, 980 nm, 1000 nm, 1020 nm, 1040 nm, 1060 nm, 1080 nm, 1100 nm, 1120 nm, 1140 nm, 1160 nm, 1180 nm, 1200 nm, 1220 nm, 1240 nm, 1260 nm, 1280 nm, 1300 nm, 1320 nm, 1340 nm, 1360 nm, 1380 nm, 1400 nm, 1420 nm, 1440 nm, 1460 nm, 1480 nm, 1500 nm, 1600 nm, 1700 nm, 1800 nm, 1900 nm, 2000 nm, 2500 nm, 3000 nm, 3500 nm, 4000 nm, 4500 nm, or 5000 nm, or within 5 nm, 10 nm, 20 nm, 30 nm, 40 nm, 50 nm, 60 nm, 70 nm, 80 nm, 90 nm, or 100 nm of any of these values, or within a range bounded by any two of these values (e.g., 400-500 nm or 960-980 nm). Some particular ranges considered herein for photoluminescence peaks include 300-500 nm, 300-1500 nm, 500-1000 nm, 500-1500 nm, 435-445 nm, 430-450 nm, 475-525 nm, 1050-1150 nm, 970-980 nm, and 970-1000 nm. In some embodiments, the metal oxide particles exhibit a photoluminescence peak above 500 nm, 800 nm, 1000 nm, 1200 nm, or 1500 nm.

In particular embodiments, the metal oxide particles possess a photoluminescence peak characterized by a full-width half maximum (FWHM) value of about or less than 20 nanometers (20 nm). In other embodiments, the metal oxide particles possess a photoluminescence peak characterized by a FWHM value of about or greater than 20 nm. In different embodiments, the metal oxide particles possess a photoluminescence peak characterized by a FWHM value of about or at least, or above, or less than 20 nm, 40 nm, 60 nm, 80 nm, 100 nm, 150 nm, 200 nm, 250 nm, 300 nm, 350 nm, 400 nm, 450 nm, 500 nm, 600 nm, 700 nm, 800 nm, 900 nm, 1,000 nm, 1,100 nm, and 1,200 nm. In yet other embodiments, the metal oxide particles possess a photoluminescence peak having a FWHM value of about or less than 15 nm, 10 nm, 8 nm, or 5 nm.

The non-oxide precursor particles can be produced by any method known in the art, including abiotic and microbial-mediated processes. Some examples of physical abiotic processes include advanced epitaxial, ion implantation, and lithographic techniques. Some examples of chemical abiotic processes include arrested precipitation in solution, synthesis in structured media, high temperature pyrolysis, and sonochemical methods. For example, as known in the art, cadmium selenide can be synthesized by arrested precipitation in solution by reacting dialkylcadmium (i.e., R₂Cd) and trioctylphosphine selenide (TOPSe) precursors in a solvent at elevated temperatures. The microbial synthesis of semiconductor nanoparticles is well known, as generally described in, for example, P. R. Smith, et al., J. Chem. Soc., Faraday Trans., 94(9), 1235-1241 (1998) and C. T. Dameron, et al., Nature, 338: 596-7, (1989), and U.S. Application Pub. No. 2010/0330367.

In particular embodiments, the non-oxide precursor particles are produced by a microbial synthesis method. In the method, a precursor chalcophile metal component (i.e., one that can form semiconducting chalcogenide compounds) and a precursor non-metal component (i.e., “non-metal component”) are processed by anaerobic microbes in a manner that produces non-oxide semiconductor particles. As the precursor metal and non-metal components are combined to make the non-oxide particles, it is understood that, generally, none of the precursor components are equivalent in composition to the particle composition.

For microbial production of non-oxide precursor particles, a precursor metal component containing one or more types of metals in ionic form, particularly as described above, are provided to microbes as a nutritive source. The one or more metals are typically in the form of a salt or coordination compound, or a colloidal hydrous metal oxide or mixed metal oxide, wherein “compound” as used herein also includes a “material” or “polymer”. Some examples of precursor metal compounds applicable herein as microbial nutritive sources include the metal halides (e.g., CuCl₂, CdCl₂, ZnCl₂, ZnBr₂, GaCl₃, InCl₃, FeCl₂, FeCl₃, SnCl₂, and SnCl₄), metal nitrates (e.g., Cd(NO₃)₂, Ga(NO₃)₃, In(NO₃)₃, and Fe(NO₃)₃), metal perchlorates, metal carbonates (e.g., CdCO₃), metal sulfates (e.g., CdSO₄, FeSO₄, and ZnSO₄), metal oxides (e.g., Fe₂O₃, CdO, Ga₂O₃, In₂O₃, ZnO, SnO, SnO₂), metal hydroxides (e.g., Fe(OH)₃ and Zn(OH)₂), metal oxyhydroxides (e.g., FeOOH, or FeO(OH), and their alternate forms), metal-EDTA complexes, metal amines (e.g., metal alkylamine, piperidine, pyridine, or bipyridine salt complexes), metal carboxylates (e.g., cadmium acetate), and metal acetylacetonate (i.e., metal-acac) complexes.

One or more dopant species can be included in the microbial precursor metal component in order to likewise dope the resulting non-oxide particles. The dopant can be any metal or non-metal species, such as any of the metal and non-metal species described above. In some embodiments, the dopant may be or include one or more lanthanide elements, such as those selected from lanthanum (La), cerium (Ce), praseodymium (Pr), neodymium (Nd), samarium (Sm), europium (Eu), gadolinium (Gd), terbium (Tb), dysprosium (Dy), holmium (Ho), erbium (Er), thulium (Tm), ytterbium (Yb), and lutetium (Lu). Generally, the dopant is present in an amount of less than 0.5 molar percent of the resulting particles, or in different embodiments, less than or up to 0.4, 0.3, 0.2, 0.1, 0.05, 0.02, or 0.01 molar percent of the resulting particles. Some examples of doped compositions include ZnS:Ni, wherein Ni functions as a dopant, as described in, for example, Bang et al., Advanced Materials, 20:2599-2603 (2008), ZnₓCd₁₋ₓS doped compositions, as described in Wang et al., Journal of Physical Chemistry C 112:16754-16758 (2008), and ZnS:Mn and ZnS:Cu compositions, as described in Song et al., Journal of Physics and Chemistry of Solids, 69:153-160 (2008). In other embodiments, a dopant is excluded, or alternatively, one or more of any of the generic or specific dopants described above are excluded.

When two or more metals are used as precursors, the molar ratio of metal ions can be adjusted such that a particular molar ratio of metals is provided in the microbial particle product. Typically, the molar ratio of metal ions in the metal component is the molar ratio of metals found in the non-oxide particle product. However, the molar ratio of metals in the product may, in several embodiments, differ from the molar ratio of metals in the metal component. In a particular embodiment, a desired molar ratio of metals is achieved in the non-oxide particle product by suitable adjustment of metal ratios in the precursor metal component.

The total metal concentration in the microbial nutritive solution should be below a concentration at which the metals are toxic to the microbes being used. Typically, the total metal concentration is no more than 100 mM. In different embodiments, the total metal concentration may preferably be no more than 90 mM, 80 mM, 70 mM, 60 mM, 50 mM, 40 mM, 30 mM, 20 mM, 15 mM, 10 mM, 5 mM, 1 mM, 0.5 mM, or 0.1 mM, or within a range resulting from any two of the above exemplary values.

The precursor non-metal component provides the resulting non-oxide particle composition with one or more chalcogen or pnictogen non-metals, e.g., S, Se, Te, N, P, As, Sb, or Bi. The non-metal component can include any suitable form of these non-metals, including, for example, the elemental or compound forms of these non-metals.

In a first embodiment of the microbial synthesis method, the non-metal component includes a source of sulfur. The source of sulfur can be, for example, elemental sulfur)(S⁰) or a sulfur-containing compound. In one instance, the sulfur-containing compound is an inorganic sulfur-containing compound. Some examples of inorganic sulfur-containing compounds include the inorganic sulfates (e.g., Na₂SO₄, K₂SO₄, MgSO₄, (NH₄)₂SO₄, H₂SO₄, or a metal sulfate), the inorganic sulfites (e.g., Na₂SO₃, H₂SO₃, or (NH₄)₂SO₃), inorganic thiosulfates (e.g., Na₂S₂O₃ or (NH₄)₂S₂O₃), sulfur dioxide, peroxomonosulfate (e.g., Na₂SO₅ or KHSO₅), and peroxodisulfate (e.g., Na₂S₂O₈, K₂S₂O₈, or (NH₄)₂S₂O₈). In another instance, the sulfur-containing compound is an organosulfur (i.e., organothiol or organomercaptan) compound. The organosulfur compound contains at least one hydrocarbon group and is typically characterized by the presence of at least one sulfur-carbon bond. Some examples of suitable organosulfur compounds include the hydrocarbon mercaptans (e.g., methanethiol, ethanethiol, propanethiol, butanethiol, thiophenol, ethanedithiol, 1,3-propanedithiol, 1,4-butanedithiol, thiophene), the alcohol-containing mercaptans (e.g., 2-mercaptoethanol, 3-mercaptopropanol, 4-mercaptophenol, and dithiothreitol), the mercapto-amino acids (e.g., cysteine, homocysteine, methionine, thioserine, thiothreonine, and thiotyrosine), mercapto-peptides (e.g., glutathione), the mercapto-pyrimidines (e.g., 2-thiouracil, 6-methyl-2-thiouracil, 4-thiouracil, 2,4-dithiouracil, 2-thiocytosine, 5-methyl-2-thiocytosine, 5-fluoro-2-thiocytosine, 2-thiothymine, 4-thiothymine, 2,4-dithiothymine, and their nucleoside and nucleotide analogs), the mercapto-purines (e.g., 6-thioguanine, 8-thioadenine, 2-thioxanthine, 6-thioxanthine, 6-thiohypoxanthine, 6-thiopurine, and their nucleoside and nucleotide analogs), the thioethers (e.g., dimethylsulfide, diethylsulfide, diphenylsulfide, biotin), the disulfides (e.g., cystine, lipoic acid, diphenyl disulfide, iron disulfide, and 2-hydroxyethyldisulfide), the thiocarboxylic acids (e.g., thioacetic acid), the thioesters, the sulfonium salts (e.g., trimethylsulfonium or diphenylmethylsulfonium chloride), the sulfoxides (e.g., dimethylsulfoxide), the sulfones (e.g., dimethylsulfone), thioketones, thioamides, thiocyanates, isothiocyanates, thiocarbamates, dithiocarbamates, and trialkylphosphine sulfide (e.g., trioctylphosphine sulfide), thiourea compounds, or any of the inorganic sulfur-containing compounds, such as those enumerated above, which have been modified by inclusion of a hydrocarbon group. In particular embodiments, the organosulfur compound includes a sulfur-containing nucleic base (i.e., S-nucleobase), such as any of the mercapto-pyrimidines and mercapto-purines described above.

In a second embodiment of the microbial synthesis method, the non-metal component includes a selenium-containing compound. The source of selenium can be, for example, elemental selenium)(Se⁰) or a selenium-containing compound. In one instance, the selenium-containing compound is an inorganic selenium-containing compound. Some examples of inorganic selenium-containing compounds include the inorganic selenates (e.g., Na₂SeO₄, K₂SeO₄, MgSeO₄, (NH₄)₂SeO₄, H₂SeO₄, or a metal selenate), the inorganic selenites (e.g., Na₂SeO₃, H₂SeO₃, or (NH₄)₂SeO₃), inorganic selenosulfates (e.g., Na₂SSeO₃ or (NH₄)₂SSeO₃), selenium dioxide, and selenium disulfide. In another instance, the selenium-containing compound is an organoselenium compound. The organoselenium compound contains at least one hydrocarbon group and is typically characterized by the presence of at least one selenium-carbon bond. Some examples of suitable organoselenium compounds include the hydrocarbon selenols (e.g., methaneselenol, ethaneselenol, n-propaneselenol, isopropaneselenol, and selenophenol (benzeneselenol)), the seleno-amino acids (e.g., selenocysteine, selenocystine, selenohomocysteine, selenomethionine), the selenopyrimidines (e.g., 2-selenouracil, 6-methyl-2-selenouracil, 4-selenouracil, 2,4-diselenouracil, 2-selenocytosine, 5-methyl-2-selenocytosine, 5-fluoro-2-selenocytosine, 2-selenothymine, 4-selenothymine, 2,4-diselenothymine, and their nucleoside and nucleotide analogs), the selenopurines (e.g., 6-selenoguanine, 8-selenoadenine, 2-selenoxanthine, 6-selenoxanthine, 6-selenohypoxanthine, 6-selenopurine, and their nucleoside and nucleotide analogs), the selenides (e.g., dimethylselenide, diethylselenide, and methylphenylselenide), the diselenides (e.g., dimethyldiselenide, diethyldiselenide, and diphenyldiselenide), the selenocarboxylic acids (e.g., selenoacetic acid, selenopropionic acid), the selenosulfides (e.g., dimethylselenosulfide), the selenoxides (e.g., dimethylselenoxide and diphenylselenoxide), the selenones, the selenonium salts (e.g., dimethylethylselenonium chloride), the vinylic selenides, selenopyrylium salts, trialkylphosphine selenide (e.g., trioctylphosphine selenide, i.e., TOPSe), selenourea compounds, or any of the inorganic selenium-containing compounds, such as those enumerated above, which have been modified by inclusion of a hydrocarbon group. In particular embodiments, the organoselenium compound includes a selenium-containing nucleic base (i.e., Se-nucleobase), such as any of the selenopyrimidines and selenopurines described above.

In a third embodiment of the microbial synthesis method, the non-metal component includes a tellurium-containing compound. The source of tellurium can be, for example, elemental tellurium)(Te⁰) or a tellurium-containing compound. In one instance, the tellurium-containing compound is an inorganic tellurium-containing compound. Some examples of inorganic tellurium-containing compounds include the inorganic tellurates (e.g., Na₂TeO₄, K₂TeO₄, MgTeO₄, (NH₄)₂TeO₄, H₂TeO₄, H₆TeO₆, or a metal tellurate), the inorganic tellurites (e.g., Na₂TeO₃), and tellurium dioxide. In another instance, the tellurium-containing compound is an organotellurium compound. The organotellurium compound contains at least one hydrocarbon group and is typically characterized by the presence of at least one tellurium-carbon bond. Some examples of suitable organotellurium compounds include the hydrocarbon tellurols (e.g., methanetellurol, ethanetellurol, n-propanetellurol, isopropanetellurol, and tellurophenol (benzenetellurol)), the telluro-amino acids (e.g., tellurocysteine, tellurocystine, tellurohomocysteine, telluromethionine), the telluropyrimidines and their nucleoside and nucleotide analogs (e.g., 2-tellurouracil), the telluropurines and their nucleoside and nucleotide analogs, the tellurides (e.g., dimethyltelluride, diethyltelluride, and methylphenyltelluride), the ditellurides (e.g., dimethylditelluride, diethylditelluride, and diphenylditelluride), the telluroxides (e.g., dimethyltelluroxide and diphenyltelluroxide), the tellurones, the telluronium salts, the vinylic tellurides, telluropyrylium salts, tellurourea compounds, 24-telluracholestanol, or any of the inorganic tellurium-containing compounds, such as those enumerated above, which have been modified by inclusion of a hydrocarbon group. In particular embodiments, the organotellurium compound includes a tellurium-containing nucleic base (i.e., Te-nucleobase), such as any of the telluropyrimidines and telluropurines described above.

In a fourth embodiment of the microbial synthesis method, the non-metal component includes an arsenic-containing compound. In one instance, the arsenic-containing compound is an inorganic arsenic-containing compound. Some examples of inorganic arsenic-containing compounds include the inorganic arsenates (e.g., Na₃AsO₄, Na₂HAsO₄, NaH₂AsO₄, H₃AsO₄, Mg₃(AsO₄)₂, 1-arseno-3-phosphoglycerate, or a transition metal arsenate), inorganic arsenites (e.g., Na₃AsO₃, Na₂HAsO3, NaH₂AsO₃, H₃AsO₃, Ag₃AsO₃, Mg₃(AsO₃)₂), and arsenic oxides (e.g., As₂O₃ and As₂O₅), and arsenous carbonate (i.e., As₂(CO₃)₃). In another instance, the arsenic-containing compound is an organoarsine compound. The organoarsine compound is characterized by the presence of at least one hydrocarbon group and at least one arsenic atom. Some examples of suitable organoarsine compounds include the hydrocarbon arsines (e.g., trimethylarsine, triethylarsine, triphenylarsine, arsole, and 1,2-bis(dimethylarsino)benzene), arsenic-derivatized sugars (e.g., glucose 6-arsenate), arsonic acids (e.g., phenylarsonic acid, 4-aminophenylarsonic acid, 4-hydroxy-3-nitrobenzenearsonic acid, 2,3,4-trihydroxybutylarsonic acid, arsonoacetic acid, diphetarsone, diphenylarsinic acid, and 3-arsonopyruvate), arseno-amino acids and their derivatives (e.g., 3-arsonoalanine, arsenophenylglycine, and arsenate tyrosine), organoarsine oxides (e.g., methylarsine oxide, 4-aminophenylarsenoxide, oxophenylarsine, and oxophenarsine), 10,10′-oxybis-10H-phenoxarsine, 1-arseno-3-phosphoglycerate, arsenobetaine, arsenocholine, arsenotriglutathione, or any of the inorganic arsenic-containing compounds, such as those enumerated above, which have been modified by inclusion of a hydrocarbon group.

Preferably, in the microbial synthesis method, the non-metal compound is not a reduced sulfide (e.g., Na₂S, K₂S, H₂S, or (NH₄)₂S), reduced selenide (e.g., H₂Se or (NH₄)₂Se), reduced telluride (e.g., H₂Te or (NH₄)₂Te), or reduced arsenide compound. As known in the art, such reduced compounds have a propensity for precipitating various metals from solution. Since direct reaction of the non-metal compound and metal to form a precipitate is preferably avoided in the method, a reduced non-metal compound is preferably used under conditions where an adverse reaction or precipitation does not occur.

The anaerobic microbes considered herein for production of non-oxide precursor particles are any microbes known in the art capable of forming non-oxide particles from one or more types of metal ions and one or more chalcogen or pnictogen non-metals. The microbe can be, for example, a eukaryotic or procaryotic (and either unicellular or multicellular) type of microbe having this ability. Of particular relevance herein are the procaryotic organisms, which are predominantly unicellular, and are divided into two domains: the bacteria and the archaea. The microbes can be, in addition, fermentative, metal-reducing, dissimilatory, sulfate-reducing, thermophilic, mesophilic, psychrophilic, or psychrotolerant. The microbes are preferably those capable of directly reducing (i.e., without the use of chemical means) a sulfur-containing, selenium-containing, tellurium-containing, or arsenic-containing compound to, respectively, a sulfide (i.e., S²⁻)-containing, selenide (i.e., Se²⁻)-containing, telluride (i.e., Te²⁻) containing, or arsenide (i.e., As²⁻)-containing compound, such as H₂S or a salt thereof. In some embodiments, the microbes reduce the sulfur-, selenium-, tellurium-, or arsenic-containing compound without intermediate production of, respectively, elemental sulfur, selenium, tellurium, or arsenic. In other embodiments, the microbes reduce the sulfur-, selenium-, tellurium-, or arsenic-containing compound with intermediate production of, respectively, elemental sulfur, selenium, tellurium, or arsenic.

In one embodiment of the microbial synthesis method for production of non-oxide precursor particles, the microbes considered herein are thermophilic, i.e., those organisms capable of thriving at temperatures of at least about 40° C. (and more typically, at least 45° C. or 50° C.) and up to about 100° C. or higher temperatures. Preferably, the thermophilic microbes are either bacteria or archaea, and particularly, those possessing an active hydrogenase system linked to high energy electron carriers.

A group of thermophilic bacteria particularly considered herein for the microbial synthesis of non-oxide precursor particles are the species within the genus Thermoanaerobacter. A particular species of Thermoanaerobacter considered herein is Thermoanaerobacter strain TOR-39, a sample of which was deposited with the American Type Culture Collection (10801 University Blvd., Manassas, Va. 20010) on Sep. 7, 2001 as accession number PTA-3695. Strain TOR-39 is a thermophile that grows optimally at temperatures from about 65 to 80° C. The conditions needed to grow and maintain this strain, including basal medium, nutrients, vitamins, and trace elements are detailed in U.S. Pat. No. 6,444,453, the entire contents of which are incorporated herein by reference. Some particular strains of Thermoanaerobacter ethanolicus particularly considered herein include T. ethanolicus strain Cl and T. ethanolicus strain M3.

Another group of thermophilic bacteria particularly considered herein for the production of non-oxide precursor particles are the species within the class Thermococci. An order of Thermococci particularly considered herein is Thermococcales. A family of Thermococcales particularly considered herein is Thermococcaceae. A genus of Thermococcaceae particularly considered herein is Thermococcus. A species of Thermococcus particularly considered herein is Thermococcus litoralis.

Another group of thermophilic bacteria particularly considered herein for the production of non-oxide precursor particles are the species within the genus Thermoterrabacterium. A species of Thermoterrabacterium particularly considered herein is Thermoterrabacterium ferrireducens, and particularly, strain JW/AS-Y7.

Still another group of thermophilic bacteria particularly considered herein for the production of non-oxide precursor particles are the species within the phylum Deinococcus-Thermus. A class of Deinococcus-Thermus particularly considered herein is Deinococci. An order of Deinococci particularly considered herein is Thermales. A genus of Thermales particularly considered herein is Thermus. A species of Thermus particularly considered herein is Thermus sp. strain SA-01.

Other thermophilic bacteria particularly considered herein for the production of non-oxide precursor particles include thermophilic species within any of the genera Thermoanaerobacterium (e.g., T. thermosulfurigenes, T. polysaccharolyticum, T. zeae, T. aciditolerans, and T. aotearoense), Bacillus (e.g., B. infernos), Clostridium (e.g., C. thermocellum), Anaerocellum (e.g., A. thermophilum), Dictyoglomus (e.g., D. thermophilum), and Caldicellulosiruptor (e.g., C. acetigenus, C. hydrothermalis, C. kristjanssonii, C. kronotskiensis, C. lactoaceticus, C. owensensis, and C. saccharolyticus).

In another embodiment, the microbes considered herein for the production of non-oxide precursor particles are mesophilic (e.g., organisms thriving at moderate temperatures of about 15-40° C.) or psychrophilic (e.g., organisms thriving at less than 15° C.). As used herein, the term “psychrophilic” also includes “psychrotolerant”. Psychrophilic bacteria are typically found in deep marine sediments, sea ice, Antarctic lakes, and tundra permafrost. Some examples of such microbes include species within the genera Shewanella (e.g., S. alga strain PV-1, S. alga, PV-4, S. pealeana, W3-7-1, S. gelidimarina, and S. frigidimarina), Clostridium (e.g., C. frigoris, C. lacusfryxellense, C. bowmanii, C. psychrophilum, C. laramiense, C. estertheticum, and C. schirmacherense), Bacillus (e.g., B. psychrosaccharolyticus, B. insolitus, B. globisporus, B. psychrophilus, B. cereus, B. subtilis, B. circulans, B. pumilus, B. macerans, B. sphaericus, B. badius, B. licheniformis, B. firmus, B. globisporus, and B. marinus), and Geobacter (e.g., G. sulfurreducens, G. bemidjiensis, and G. psychrophilus). Of particular interest are those strains capable of anaerobic growth with nitrate as an electron acceptor.

In yet another embodiment, the microbes considered herein for the production of non-oxide precursor particles are sulfur-reducing (e.g., sulfate- or sulfite-reducing) microbes. In a preferred embodiment, the sulfur-reducing microbes are one or more species selected from Desulfovibrio (e.g., D. desulfuricans, D. gigas, D. salixigens, and D. vulgaris), Desulfolobus (e.g., D. sapovorans and D. propionicus), Desulfotomaculum (e.g., D. thermocisternum, D. thermobenzoicum, D. auripigmentum, D. nigrificans, D. orientis, D. acetoxidans, D. reducens, and D. ruminis), Desulfomicrobium (e.g., D. aestuarii, D. hypogeium, and D. salsuginis), Desulfomusa (e.g., D. hansenii), Thermodesulforhabdus (e.g., T. norvegica) the order Desulfobacterales, and more particularly, the family Desulfobacteraceae, and more particularly, the genera Desulfobacter (e.g., D. hydrogenophilus, D. postgatei, D. giganteus, D. halotolerans, and D. vibrioformis), Desulfobacterium (e.g., D. indolicum, D. anilini, D. autotrophicum, D. catecholicum, D. cetonicum, D. macestii, D. niacini, D. phenolicum, D. vacuolatum), Desulfobacula (e.g., D. toluolica and D. phenolica), Desulfobotulus (D. sapovorans and D. marinus), Desulfocella (e.g., D. halophila), Desulfococcus (e.g., D. multivorans and D. biacutus), Desulfofaba (e.g., D. gelida and D. fastidiosa), Desulfofrigus (e.g., D. oceanense and D. fragile), Desulfonema (e.g., D. limicola, D. ishimotonii, and D. magnum), Desulfosarcina (e.g., D. variabilis, D. cetonica, and D. ovata), Desulfospira (e.g., D. joergensenii), Desulfotalea (e.g., D. psychrophila and D. arctica), and Desulfotignum (D. balticum, D. phosphitoxidans, and D. toluenicum). Several of the sulfur-reducing microbes are either thermophilic or mesophilic. The sulfur-reducing microbes may also be psychrophilic or psychrotolerant.

In still other embodiments, the microbes considered herein for the production of non-oxide precursor particles are selenium-reducing (e.g., selenate-, selenite-, or elemental selenium-reducing), tellurium-reducing (e.g., tellurite-, tellurite-, or elemental tellurium-reducing), or arsenic-reducing (e.g., arsenate- or arsenite-reducing). In one embodiment, the selenium-, tellurium-, or arsenic-reducing microbe is one of the sulfur-reducing microbes described above. In another embodiment, the selenium- or tellurium-reducing microbe is selected from other microbes not described above, e.g., Thauera selenatis, Sulfospirillum barnesii, Selenihalanerobacter shriftii, Bacillus selenitireducens, Pseudomonas stutzeri, Enterobacter hormaechei, Klebsiella pneumoniae, and Rhodobacter sphaeroides. In yet another embodiment, the arsenic-reducing microbe is selected from any of the microbes described above, or in particular, from Sulfurospirillum arsenophilum or Geospirillum arsenophilus. It will also be appreciated that, in addition to the exemplary microorganisms listed above, other types of cultures, including mixed microbial cultures or uncharacterized microbial cultures from natural environments, and the like, may also be used in the invention. For example, cultures not yet characterized from natural hot springs where various metals are known to be present can demonstrate suitably high metal-reducing or selenium-reducing activity to carry out the inventive methods even though the exact species or genus of the microbes may be unknown and more than one species or genus may be present in said culture.

The microbes can also be dissimilatory iron-reducing bacteria. Such bacteria are widely distributed and include some species in at least the following genera: Bacillus, Deferribacter, Desulfuromonas, Desulfuromusa, Ferrimonas, Geobacter, Geospirillum, Geovibrio, Pelobacter, Sulfolobus, Thermoanaerobacter, Thermoanaerobium, Thermoterrabacterium, and Thermus.

The choice of microbe generally involves trade-offs relating to cost, efficiency, and properties of the non-oxide particle product. For example, thermophiles may be preferred when more product per unit of time is the primary consideration, since a high temperature process generally produces product at a faster rate. Conversely, psychrophilic or psychrotolerant microbes may be preferred in a case where one or more improved characteristics are of primary consideration, and where the improved characteristics are afforded to the product by virtue of the cooler process.

The microbes used in the method described herein for the production of non-oxide precursor particles can be obtained and cultured by any of the methods known in the art. Some of the general processes by which such bacteria may be used are taught in U.S. Pat. Nos. 6,444,453 and 7,060,473, the entire disclosures of which are incorporated herein by reference. The isolation, culturing, and characterization of thermophilic bacteria are described in, for example, T. L. Kieft et al., “Dissimilatory Reduction of Fe(III) and Other Electron Acceptors by a Thermus Isolate,” Appl. and Env. Microbiology, 65 (3), pp. 1214-21 (1999). The isolation, culture, and characterization of several psychrophilic bacteria are described in, for example, J. P. Bowman et al., “Shewanella gelidimarina sp. nov. and Shewanella frigidimarina sp. nov., Novel Antarctic Species with the Ability to Produce Eicosapentaenoic Acid (20:5ω3) and Grow Anaerobically by Dissimilatory Fe(III) Reduction,” Int. J. of Systematic Bacteriology 47 (4), pp. 1040-47 (1997). The isolation, culture, and characterization of mesophilic bacteria are described in, for example, D. R. Lovley et al., “Geobacter metallireducens gen. nov. sp. nov., a microorganism capable of coupling the complete oxidation of organic compounds to the reduction of iron and other metals,” Arch. Microbiol., 159, pp. 336-44 (1993), the disclosure of which is incorporated herein by reference in its entirety.

The culture medium for sustaining the microbes for the production of non-oxide precursor particles can be any of the known aqueous-based media known in the art useful for this purpose. The culture medium may also facilitate growth of the microbes. As is well known in the art, the culture medium includes such components as nutrients, trace elements, vitamins, and other organic and inorganic compounds, useful for the sustainment or growth of microbes.

In the microbial process for the production of non-oxide precursor particles, the microbes are provided with at least one electron donor. An electron donor is any compound or material capable of being oxidatively consumed by the microbes such that donatable electrons are provided to the microbes by the consumption process. The produced electrons are used by the microbes to reduce one or more non-metal compounds and/or metal ions.

In one embodiment, the electron donor includes one or more carboxylate-containing compounds that can be oxidatively consumed by the microbes. Some examples of suitable carboxylate-containing compounds include formate, acetate, propionate, butyrate, oxalate, malonate, succinate, fumarate, glutarate, lactate, pyruvate, glyoxylate, glycolate, and citrate.

In another embodiment, the electron donor includes one or more sugars (i.e., saccharides, disaccharides, oligosaccharides, or polysaccharides) that can be oxidatively consumed by the microbes. Some examples of suitable sugars include glucose, fructose, sucrose, galactose, maltose, mannose, arabinose, xylose, lactose, and disaccharides therefrom, oligosaccharides therefrom, or polysaccharides therefrom.

In another embodiment, the electron donor includes one or more inorganic species that can be oxidatively consumed by the microbes. The inorganic species can be, for example, an oxidizable gas, such as hydrogen or methane. Such gases can be oxidized by hydrogen-consuming or methane-consuming microbes which have the capacity to reduce one or more metals or non-metal compounds by the produced electrons.

For the microbial production of non-oxide precursor particles, the five reaction components described above (i.e., anaerobic microbes, culture medium, metal component, non-metal component, and electron donor component) are combined in a suitable container and subjected to conditions (e.g., temperature, pH, and reaction time) suitable for producing the non-oxide particles from the reaction components. In one embodiment, the container for holding the reaction components is simple by containing no more than container walls, a bottom, and a lid. In another embodiment, the container is more complex by including additional features, such as inlet and outlet elements for gases, liquids, or solids, one or more heating elements, nanoparticle separation features (e.g., traps or magnets), one or more agitating elements, fluid recirculating elements, electronic controls for controlling one or more of these or other conditions, and so on.

The components may be combined in any suitable manner. For example, each of the five reaction components or a combination thereof (e.g., the anaerobic microbes and cell culture) may be prepared before the components are combined, or alternatively, obtained in a pre-packaged form before the components are combined. When components or combinations thereof are provided in package form, the packaged forms may be designed to be used in their entireties, or alternatively, designed such that a portion of each is used (e.g., as aliquots of a concentrate).

The method for the microbial production of non-oxide precursor particles is generally practiced by subjecting the combined components to conditions that induce the formation of non-oxide precursor particles therefrom. Some of the conditions that can affect formation of non-oxide particles from the combined components include temperature, reaction time, precursor metal concentration, pH, and type of microbes used. In some embodiments, the reaction conditions may not require any special measures other than combining the reaction components at room temperature (e.g., 15-25° C.) and waiting for particles to grow over a period of time. In other embodiments, the combined reaction components are, for example, either heated, cooled, or modified in pH, in order to induce non-oxide particle formation.

When thermophilic microbes are used, the temperature at which the reaction is conducted can preferably be at least, for example, 40° C., 45° C., 50° C., 55° C., 60° C., 65° C., 70° C., 75° C., 80° C., 85° C., or 90° C. depending on the type of thermophilic microbes being used. Any range resulting from any two of the foregoing values is also contemplated herein. When mesophilic microbes are used, the temperature can preferably be at least 15° C., 20° C., 25° C., or 30° C., and up to any of the temperatures given above for thermophilic microbes. When psychrophilic microbes are used, the temperature at which the reaction is conducted can preferably be less than, for example, 40° C., or at or less than 35° C., 30° C., 25° C., 20° C., 15° C., 10° C., 5° C., 0° C., or −5° C., or any range resulting from any two of the foregoing values. It is to be appreciated that, even though different exemplary temperatures have been given for each type of microbe, each type of microbe may be capable of thriving in temperatures well outside the typical temperatures given above. For example, a thermophilic microbe may also be capable of thriving to a useful extent at temperatures below 40° C. where mesophilic microbes traditionally thrive; or mesophilic or thermophilic microbes may be capable of thriving to a useful extent at temperatures below 15° C. (i.e., by being psychrotolerant in addition to mesophilic or thermophilic). Particularly when employing Thermoanaerobacter sp. strain TOR-39, the temperature is preferably maintained between about 45° C. and 75° C.

The reaction (incubation) time is the period of time that the combined reaction components are subjected to reaction conditions necessary for producing non-oxide precursor particles. The reaction time is very much dependent on the other conditions used, as well as the characteristics desired in the non-oxide particle product. For example, shorter reaction times (e.g., 1-60 minutes) may be used at elevated temperature conditions whereas longer reaction times (e.g., 1-7 days, or 1-3 weeks) may be used at lower temperatures to obtain a similar yield of product. Typically, shorter reaction times produce smaller particles than particles produced using longer reaction times under the same conditions. The incubation may be, for example, between 3 and 30 days, depending on the amount and size of the particle product desired.

The pH of the microbial nutritive solution can also be suitably adjusted. Generally, when using thermophilic bacteria, the pH value is preferably within the range of 6.5-9. For example, particularly when employing Thermoanaerobacter sp. strain TOR-39, the pH is preferably maintained at a level between about 6.9 and 7.5. In different embodiments, depending on the microbe and other conditions, the pH is preferably acidic by being less than 7 (e.g., a pH of or less than 6.5, 6.0, 5.5, 5.0, 4.5, 4.0, 3.5, 3.0, 2.5, 2.0, 1.5, 1.0, or a range resulting from any two of these values), or preferably alkaline by being above 7 (e.g., a pH of or greater than 7.5, 8.0, 8.5, 9.0, 9.5, 10, 10.5, 11, 11.5, or a range resulting from any two of these values), or preferably approximately neutral by having a pH of about 7, e.g., 6.5-7.5.

In addition to selecting reaction conditions (e.g., temperature, reaction time, and pH) on the basis of permitting or inducing the formation of non-oxide precursor particles, the reaction conditions can also be selected for numerous other purposes, including to modify or optimize the product yield, production efficiency, particle size or size range, particle composition or phase (e.g., crystalline vs. semicrystalline vs. amorphous), or particle morphology. For example, lower reaction temperatures may be employed to provide a more pure or single-crystalline product.

Once the non-oxide precursor particles are microbially produced, they are isolated (i.e., separated) from the reaction components and byproducts formed by the reaction products. Any method known in the art for separation of particles from reaction components can be used herein.

In one embodiment, the non-oxide particles are separated from the microbial reaction components by allowing the particles to settle to the bottom of the container and then decanting the liquid medium or filtering off the particles. This settling may be accomplished with or without centrifugation. When centrifugation is used, the centrifugal (i.e., “g” force) causes settling of denser particles to the bottom or distal end of the spun containers. The collected particles may be washed one or more times to further purify the product. The reaction container may optionally be fitted with a drain valve to allow the solid product to be removed without decanting the medium or breaking gas seals.

In another embodiment, the container in which the reaction components are housed is attached to (or includes) an external trap from which the particles can be removed. The trap is preferably in the form of a recess situated below flowing reaction solution. Particles in the flowing reaction solution are denser than the reaction solution, and hence, will settle down into the trap. The flowing reaction solution is preferably recirculated.

In another embodiment, a filter is used to trap the microbially-produced non-oxide particles. The filter can be in the form of multiple filters that trap successively smaller particles. Depending on the particle size and other variables, one or more filters that trap the non-oxide particles may contain a pore size of no more than about 0.5, 0.4, 0.3, 0.25, 0.2, 0.1, or 0.05 μm.

In yet another embodiment, in the case where the microbially-produced non-oxide particles are magnetic, a magnetic source (e.g., electromagnet or other suitable magnetic field-producing device) can be employed to collect the particles. The magnetic source can be used as the sole means of separation, or used in combination with other separation means, such as a trap or filter.

In a particular set of embodiments, the general microbial method described above is specifically directed to the preparation of particles having a CIGs-type composition. The method generally includes: (a) subjecting a combination of reaction components to conditions conducive to microbially-mediated formation of the non-oxide particles, wherein the combination of reaction components includes i) anaerobic microbes, ii) a culture medium suitable for sustaining the anaerobic microbes, iii) a metal component that includes Cu ions and at least one type of metal ion selected from In and Ga, iv) a non-metal component that includes at least one non-metal selected from S, Se, and Te, and v) one or more electron donors that provide donatable electrons to the anaerobic microbes during consumption of the electron donor by the anaerobic microbes; and (b) isolating the CIGs particles.

In another particular set of embodiments, the general microbial method described above is specifically directed to the production of non-oxide particles having a kesterite or thermoelectric composition. The method generally includes: (a) subjecting a combination of reaction components to conditions conducive to microbially-mediated formation of the non-oxide particles, wherein the combination of reaction components includes i) anaerobic microbes, ii) a culture medium suitable for sustaining the anaerobic microbes, iii) a chalcophile metal component that includes at least one chalcophile metal, iv) a non-metal component that includes at least one non-metal selected from S, Se, and Te, and v) one or more electron donors that provide donatable electrons to the anaerobic microbes during consumption of the electron donor by the anaerobic microbes; and (b) isolating the kesterite or thermoelectric particles.

In other embodiments, the invention is directed to a method for forming a component of a device which incorporates any of the above-described metal oxide particles. In particular embodiments, the metal oxide particles are deposited onto a substrate (by, for example, spray-coating, dip-coating, spin-coating, drop-casting, or inkjet printing the substrate with a solution or suspension containing the metal oxide particles), the coated substrate is typically dried and annealed, and optionally overlaid with a sealant or functional overlayer. In some embodiments, an ink-jet spraying process is used in which multiple ink-jet heads spray a multiplicity of different particle compositions Ink jet spraying methods, particularly as used in producing patterned surfaces, are described in detail in, for example, U.S. Pat. Nos. 7,572,651, 6,506,438, 6,087,196, 6,080,606, 7,615,111, 7,655,161, and 7,445,731, the contents of which are incorporated herein by reference in their entirety. In other embodiments, an ultrasonic or sonospray coating process is used. The sonospray method is described in detail in, for example, U.S. Pat. Nos. 4,153,201, 4337,896, 4,541,564, 4,978,067, 5,219,120, 7,712,680, as well as J. Kester, et al., CP394, NREL/SNL PV Prog. Rev., pp. 162-169, AIP Press, NY, 1997, the contents of which are herein incorporated by reference in their entirety. The sonospray method is a non-vacuum deposition method amenable to the manufacture of large area films, along with low processing costs. In brief, the sonospray method employs an ultrasonic nozzle that operates by use of a piezoelectric transducer that produces a high frequency motion when subjected to a high frequency electrical signal. The high frequency vibration produced by the piezoelectric material travels down a horn of the nozzle. Liquid emerging from the surface of the horn is broken into a fine spray, which is highly controllable with respect to droplet size and distribution. The deposition temperature can be any suitable temperature, but particularly for temperature-sensitive substrates, such as plastics, the deposition temperature is preferably up to or less than 200, 180, 150, 120, 100, or 80° C.

In alternative embodiments, non-oxide precursor particles are deposited onto a substrate by any of the methods described above, and the coated substrate is subjected to the oxidation process described above to convert the precursor particles to metal oxide particles that remain affixed to the substrate, thus obviating the need to deposit the metal oxide particles onto the substrate. In yet other embodiments, a multi-layer (e.g., bilayer, trilayer, etc.) coating is provided on a substrate by, for example, depositing a first layer of metal oxide particles (with optional post-annealing, fixing, or sealing), and then depositing a subsequent coating of metal oxide particles of the same or different composition.

Moreover, the single layer or multilayer being deposited may be patterned by methods known in the art (e.g., by lithographic techniques) to produce a more sophisticated electronic or photonic device. In a first set of embodiments, a patterned structure is produced by (i) subjecting a select portion of a first layer of precursor non-oxide particles to a pattern-wise pulse of thermal energy that pattern-wise (e.g., via a mask or scribing technology) oxidizes (and optionally melts or fuses) the select portion of precursor non-oxide particles to form a pattern of oxidized (and optionally, melted or fused) particles in the first layer. A patterned multilayer structure may be produced by, for example, producing a patterned first layer, as above, and then (ii) depositing a second layer of precursor non-oxide particles on the first patterned layer; and (iii) subjecting at least a portion of the second layer of precursor particles to a pulse of thermal energy to oxidize (and optionally melt or fuse) at least a portion of the second layer of precursor particles to form a pattern of oxidized (and optionally, melted or fused) particles in the second layer. Successive (e.g., third, fourth, and higher numbers) of layers may be similarly deposited. Alternatively, a first deposited layer is not patterned, while a second deposited layer is patterned, and vice-versa.

In a second set of embodiments, a patterned structure is produced by (i) producing an initial patterned layer of precursor non-oxide particles, such as provided by a selective deposition process, such as ink-jet printing or sonospray techniques, and then (ii) subjecting the patterned layer of precursor non-oxide particles to a non-pulsed or pulsed form of thermal energy that oxidizes (and optionally melts or fuses) the precursor particles to form a pattern of oxidized (and optionally, melted or fused) particles in the first layer. A patterned multilayer structure may be produced by, for example, producing a patterned first layer, as above, and then (iii) depositing a second layer of precursor particles on the patterned first layer; and (iv) subjecting at least a portion of the second layer of precursor particles to a non-pulsed or pulsed form of thermal energy that oxidizes (and optionally melts or fuses) at least a portion of the second layer of precursor particles to form a pattern of oxidized (and optionally, melted or fused) particles in the first layer.

The substrate can be useful for any applicable electronic or photonic device, such as a display, photovoltaic device (e.g., solar cell), electrode, sensor, optoelectronic device, phosphor, or electronic chip. In a first set of embodiments, the substrate is a metal substrate. Some examples of metal substrates include those composed exclusively of, or an alloy of copper, cobalt, nickel, zinc, palladium, platinum, gold, ruthenium, molybdenum, tantalum, rhodium, or stainless steel. In a second set of embodiments, the substrate is a semiconductor substrate. Some examples of semiconductor substrates include those composed exclusively of, or an alloy of silicon, germanium, indium, or tin, or an oxide, sulfide, selenide, telluride, nitride, phosphide, arsenide, or antimonide of any of these or other metals, such as of copper, zinc, or cadmium, including any of the metal oxide, metal chalcogenide, and metal pnictide compositions described above. In a third set of embodiments, the substrate is a dielectric substrate. Some general examples of dielectric substrates include ceramics, glasses, plastics, and polymers. The substrate may also have a combination of materials (e.g., metal and/or semiconductor components, along with a dielectric component). Some of these substrates, such as molybdenum-coated glass and flexible plastic or polymeric film, are particularly considered herein for use in photovoltaic applications. The photovoltaic substrate can be, for example, an absorber layer, emitter layer, or transmitter layer useful in a photovoltaic device. Other of these substrates can be used as dielectric or conductive layers in a semiconductor assembly device. Still other of these substrates (e.g., W, Ta, and TaN) may be useful as copper diffusion barrier layers, as particularly used in semiconductor manufacturing. The coating method described herein is particularly advantageous in that it can be practiced on a variety of heat-sensitive substrates (e.g., low-temperature plastic films) without damaging the substrate.

Examples have been set forth below for the purpose of illustration and to describe certain specific embodiments of the invention. However, the scope of this invention is not to be in any way limited by the examples set forth herein.

### Example 1

### Preparation of ZnO Nanoparticles

ZnS nanocrystals were synthesized by a nanofermentation technique employing Thermoanaerobacter microbes. The ZnS nanocrystals with the tailored size in the scalable process can be thermally oxidized to ZnO nanocrystals with a slight increase in the average crystallite size (ACS). The thermal treatment of the microbially-produced ZnS nanocrystal was investigated under the following atmospheres: argon gas (Ar (g), 99.999%), nitrogen gas (N₂ (g), 99.999%), and air. ZnS powder was placed in an alumina crucible, loaded into a tube furnace, and then annealed at 600° C. with a dwelling time of 2 hours and a ramping rate of 10° C./min under each atmosphere.

FIG. 1 shows the XRD patterns of the as-synthesized ZnS nanocrystals and the nanocrystals annealed in the different gases. The results of the XRD analysis of the as-synthesized ZnS indicate the diffraction crystal planes of (111), (220) and (311) in the zinc blend crystal structure with an ACS of 8.5 nm. The XRD peaks of ZnS nanocrystals annealed in the inert gases Ar (g) and N₂ (g) became narrow due to the increase of the ACS. The estimated ACS of the ZnS nanocrystals annealed in Ar (g) and N₂ (g) are 27.0 nm and 19.6 nm, respectively. The annealing of ZnS nanocrystals in air demonstrated a phase transition to ZnO as shown in the XRD pattern of FIG. 1. The XRD peaks of ZnO nanocrystals with hexagonal crystal structure were indexed to the diffraction planes of (100), (002), (101), (102), (110), (103) and (201) by matching with the JCPDS (Joint Committee on Powder Diffraction Standard) for zinc oxide with number 36-1451. The calculated ACS of the annealed ZnO was 40.3 nm.

Photoluminescence (PL) properties of the ZnS nanocrystals annealed under the different gaseous atmospheres are provided in FIG. 2. Compared to the ZnS nanocrystals annealed in inert gases, the oxidized ZnO nanocrystals show an enhancement in the relative PL intensity. The PL peak of the oxidized ZnO nanocrystals are found at 498 nm (2.49 eV), not at 376 nm (3.30 eV) due to the energy band gap of ZnO. The PL peak of the oxidized ZnO from nanofermented ZnS nanocrystals might be attributed to the recombination between the conduction band and the oxide antisite defect levels.

### Example 2

### Preparation of CuO and SnO₂ Nanoparticles

CuS and SnS precursors were microbially produced according to the process described in U.S. Patent Application Publication Nos. 2010/0330367 and 2010/0193752, the contents of which are herein incorporated by reference in their entirety. In brief, a fermentation medium that included a nutritive electron donor (e.g. glucose), thiosulfate, and thermophilic bacteria was incubated at 65° C., and then metal salts were dosed therein to produce the CuS and SnS nanoparticles. As found by X-ray diffraction analysis, as provided by the XRD patterns in FIGS. 3 and 4, the CuS and SnS precursor nanoparticles possessed an average crystallize size of 9.7±0.9 nm and 4.3±0.3 nm, respectively. The CuS and SnS precursor samples were annealed by gradually increasing the temperature to a final temperature of 800° C. in an air environment, wherein the final temperature was reached at different temperature ramping rates. For the case of CuO nanoparticles, a temperature ramping rate of 10° C./min resulted in CuO nanoparticles having a crystallite size of 29.8±3.5 nm; a temperature ramping rate of 100° C./min resulted in CuO nanoparticles having a crystallite size of 32.3±2.1 nm; and a temperature ramping rate of 500° C./min resulted in CuO nanoparticles having a crystallite size of 43.5±6.9 nm with a mixed composition containing Cu₂O due to the lack of oxidation time. For the case of SnO₂ nanoparticles, a temperature ramping rate of 10° C./min resulted in SnO₂ nanoparticles having a crystallite size of 4.8±0.1 nm; and a temperature ramping rate of 100° C./min resulted in SnO₂ nanoparticles having a crystallite size of 11.6±0.2 nm. Notably, in the case of SnS conversion to SnO₂, a surprisingly smaller change in size occurred despite the 800° C. annealing step. The foregoing results demonstrate the surprising result that the temperature ramping rate can have a pronounced effect on the size and composition of the resulting metal oxide nanoparticles. Thus, the temperature ramping rate can be carefully selected or adjusted to provide metal oxide nanoparticles of a particular size and composition.

While there have been shown and described what are at present considered the preferred embodiments of the invention, those skilled in the art may make various changes and modifications which remain within the scope of the invention defined by the appended claims.

What is claimed is:

1. A method for producing metal oxide particles, the method comprising subjecting non-oxide metal-containing particles to an oxidation step that converts the non-oxide metal-containing particles to said metal oxide particles.

2. The method of claim 1, wherein said metal oxide particles possess a size of up to about 10 microns.

3. The method of claim 1, wherein said metal oxide particles possess a size of up to 1 micron.

4. The method of claim 1, wherein said metal oxide particles possess a size of up to 100 nm.

5. The method of claim 1, wherein said metal oxide particles possess a size of up to 50 nm.

6. The method of claim 1, wherein said metal oxide particles possess a size of up to 20 nm.

7. The method of claim 1, wherein said metal oxide particles have a mono-metal oxide composition.

8. The method of claim 7, wherein the metal in said mono-metal oxide is selected from Sc, Ti, V, Cr, Mn, Fe, Co, Ni, Cu, Zn, Y, Zr, Nb, Hf, Ta, Cd, Mo, W, Ag, Pd, Pt, Ga, In, Tl, Ge, Sn, Pb, Sb, and Bi.

9. The method of claim 1, wherein said metal oxide particles have a mixed-metal oxide composition containing at least two metals.

10. The method of claim 9, wherein said at least two metals are selected from Sc, Ti, V, Cr, Mn, Fe, Co, Ni, Cu, Zn, Y, Zr, Nb, Hf, Ta, Cd, Mo, W, Ag, Pd, Pt, Ga, In, Tl, Ge, Sn, Pb, Sb, and Bi.

11. The method of claim 1, wherein said non-oxide metal-containing particles have a composition selected from metal sulfide, metal selenide, metal telluride, metal nitride, metal phosphide, metal arsenide, metal antimonide, and combinations thereof.

12. The method of claim 11, wherein said metal is selected from Zn, Cd, Cu, Fe, Co, Ni, Ga, In, Sn, Pb, Ag, Pd, Pt, and combinations thereof.

13. The method of claim 1, wherein said oxidation step employs an elevated temperature of at least 50° C. and up to 2200° C.

14. The method of claim 1, wherein said oxidation step employs a temperature of up to 120° C.

15. The method of claim 1, wherein said non-oxide metal-containing particles are deposited on a substrate prior to being subjected to the oxidation step.

16. The method of claim 15, wherein said substrate is a functional substrate on which the metal oxide particles remain for integration into an electronic or photonic device.

17. The method of claim 1, wherein said non-oxide metal-containing particles are subjected to a pulse of thermal energy having an intensity and duration of time effective for converting the non-oxide metal-containing particles to said metal oxide particles.

18. The method of claim 17, wherein the pulse has a duration of up to 1 second.

19. The method of claim 17, wherein the pulse has a duration of up to 100 milliseconds.

20. The method of claim 17, wherein the pulse has a duration of up to 100 microseconds.

21. The method of claim 1, wherein said non-oxide metal-containing particles are prepared by microbial synthesis.

22. The method of claim 21, wherein said microbial synthesis comprises:
(a) subjecting a combination of reaction components to conditions conducive to microbially-mediated formation of non-oxide metal-containing particles, wherein said combination of reaction components comprises i) anaerobic microbes, ii) a culture medium suitable for sustaining said anaerobic microbes, iii) a chalcophile metal component comprising at least one type of metal ion, iv) a non-metal component comprising at least one non-metal selected from the group consisting of S, Se, Te, and As, and v) one or more electron donors that provide donatable electrons to said anaerobic microbes during consumption of the electron donor by said anaerobic microbes; and
(b) isolating said non-oxide metal-containing particles comprised of at least one of said metal ions and at least one of said non-metals.

23. The method of claim 1, wherein said non-oxide metal-containing particles are prepared by abiotic synthesis.
